(19) United States
(12) Patent Application Publication
(10) Pub. No.: US 2007/0041933 A1
(43) Pub. Date: Feb. 22, 2007

(54) Functionally Diverse Macromolecules and Their Synthesis

(76) Inventors: Eric E. Simanek, College Station, TX (US); Emily Hollink, Ottawa (CA)

Correspondence Address: FULBRIGHT & JAWORSKI L.L.P., 600 CONGRESS AVE., SUITE 2400, AUSTIN, TX, 78701, US

(21) Appl. No.: 11/419,200

(22) Filed: May 18, 2006

Related U.S. Application Data
(60) Provisional application No. 60/706,884, filed on Aug. 10, 2005.

(51) Int. Cl. A61K 31/53; C07D 403/14

(52) U.S. Cl. 424/78.3; 514/241; 544/198; 977/754

(57) ABSTRACT

Functionally diverse macromolecules and synthetic routes for obtaining the same are disclosed. In certain embodiments, the synthesis proceeds in a divergent manner. In other embodiments, the routes rely on the differential reactivity of monomeric electrophilic triazine building blocks that display protected or unprotected groups. This diversity permits the facile introduction of a variety of diversity groups at multiple positions of these macromolecules, thereby setting the stage for further generational growth of the macromolecule and/or incorporation of other diversity groups such as biocompatible targeting groups.

### CROSS REFERENCE TO RELATED APPLICATION

This application claims the benefit of U.S. Provisional Application No. 60/706,884, filed Aug. 10, 2005, which is incorporated by reference in its entirety. The government has rights in the present invention pursuant to NIH grant (NIGMS 65460).

### BACKGROUND OF THE INVENTION

A. Field of the Invention

The present invention relates generally to synthetic routes for obtaining functional group diversity in macromolecules and to functionally diverse macromolecules. This diversity permits the facile introduction of a variety of diversity groups at multiple positions of these macromolecules, thereby setting the stage for further generational growth of the macromolecule and/or incorporation of other groups such as biocompatible targeting groups.

B. Background of the Invention

Even when reduced to trivial manipulations, the synthesis of macromolecules such as dendrimers remains laborious (Grayson et al., Chem. Rev. 2001, 101, 3819-3867; G. R. Newkome, F. Vögtle, C. N. Moorefield, Dendrimers and Dendrons: Concepts, Syntheses, Applications; VCH: New York, 2001; J. M. J. Frèchet, D. A. Tomalia, Dendrimers and Other Dendritic Polymers; John Wiley: New York, 2002) with few exceptions—notably, one-pot syntheses (Okaniwa et al., Macromolecules. 2002, 35, 6232-6238; and Rannard, J. Am. Chem. Soc. 2000, 122, 11729-1170). As applications are pursued for these macromolecular architectures, the need to execute structure-property relationships only further increases the burden of synthesis. Not surprisingly, the number of reports of libraries of macromolecules such as dendrimers are exceedingly few, and in many cases these libraries are the result of substoichiometric (with respect to the number of reactive surface groups) and statistical reactions of the periphery to yield cocktails of molecules as opposed to single-molecule chemical entities (Singh, Bioconjugate Chem. 1998, 9, 54-63; Newkome et al., Angew. Chem. Int. Ed. 1998, 37, 307-310; Newkome et al., Biotechnol. Bioeng. 1999, 61, 243-253; Baker et al., Biomed. Microdevices. 2001, 3, 61-69).

### SUMMARY OF THE INVENTION

The inventors have identified means of providing facile divergent synthetic methods for the preparation of functionally diverse macromolecules as single-molecule chemical entities. In certain non-limiting aspects, the macromolecules display a variety of diversity groups at multiple positions, thereby setting the stage for further generational growth of the macromolecule and/or incorporation of other diversity groups such as biocompatible targeting groups.

One embodiment of the invention is a method of synthesizing a macromolecule, comprising: (a) obtaining a nucleophile-bearing core including at least one nucleophilic group, and (b) reacting the core with a first monomeric electrophilic triazine including at least one substitutable group and at least one of either a protected-nucleophilic group or an unreactive group, wherein the macromolecule has one less substitutable group when compared to the first monomeric electrophilic triazine and at least one of either a protected-nucleophilic group or unreactive group. In a further embodiment, the first monomeric electrophilic triazine includes at least 2 substitutable groups, such that the macromolecule includes at least one substitutable group. In another embodiment the method further comprises reacting the macromolecule with at least one substitutable group with a first diversity group, such that at least one substitutable group is substituted with the first diversity group. In yet another embodiment, the method further comprises removing at least one protecting group of the macromolecule.

In another embodiment of the invention, the method further comprises reacting the macromolecule with a second electrophilic monomeric triazine including at least one substitutable group and at least one of either a protected-nucleophilic group or an unreactive group, wherein the macromolecule has one less substitutable group when compared to the second monomeric electrophilic triazine and at least one of either a protected-nucleophilic group or unreactive group. In a further embodiment, the method further comprises removing one or more protecting groups. In still another embodiment, the method further comprises subjecting the macromolecule to iterative steps of the methods described above, wherein the macromolecule becomes the nucleophile-bearing core of step (a). In yet another embodiment, the method further comprises subjecting the macromolecule to iterative steps of the methods described above, wherein the macromolecule becomes the nucleophile-bearing core of step (a). In another embodiment, the first monomeric electrophilic triazine includes at least one unreactive group.

In a further embodiment of the invention, the first monomeric electrophilic triazine further comprises at least one diversity group and two substitutable groups. In still another embodiment, the method further comprises reacting the macromolecule with a nucleophile-bearing group, wherein the macromolecule has one less substitutable group when compared to the first monomeric electrophilic triazaine. In yet another embodiment, the method further comprises removing one or more protecting groups from the macromolecule, while in another embodiment the first monomeric electrophilic triazine includes at least one nucleophile-bearing group and at least two substitutable groups. In another embodiment, the method further comprises reacting the macromolecule with a diversity group, wherein the macromolecule has one less substitutable group when compared to the first monomeric electrophilic triazaine. In yet another embodiment, the method further comprises removing one or more protecting groups from the macromolecule, while in another embodiment the method further comprises subjecting the macromolecule to iterative steps of other embodiments of the method.

In another embodiment, the method further comprises subjecting the macromolecule to iterative steps of other embodiments of the method, wherein the macromolecule becomes the nucleophile-bearing core of those embodiments. In some embodiments, the method further comprises subjecting the macromolecule to iterative steps of other embodiments of the method, wherein the macromolecule becomes the nucleophile-bearing group of those embodiments. In still other embodiments, the method further comprises subjecting the macromolecule to iterative steps of other embodiments, wherein the macromolecule becomes the nucleophile-bearing core of those embodiments.

In a further embodiment, the first monomeric electrophilic triazine includes at least 2 or at least 4 nucleophilic groups. In another embodiment, the nucleophile-bearing core including at least one nucleophilic group comprises: 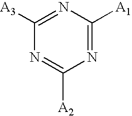
wherein A1 is a first nucleophile-bearing group and, A2 and A3 are selected from a group consisting of a second nucleophile-bearing group and a third nucleophilic group, and an unreactive group. In yet another embodiment, any one or more of A1-A3 is an amine-bearing group. In still another embodiment, the amine-bearing group is selected from a group consisting of an —NH2-bearing group and an —R—NH2-bearing group, wherein R is selected from the group consisting of a hydrocarbon-containing group, a secondary or tertiary amine-containing group, an oxo-containing group, a thiol-containing group, an amido-containing group, and any combination of one or more of these groups. In another embodiment, the amine-bearing group is a secondary amine-bearing group. In still another embodiment, the secondary amine-bearing group is a cycloalkylamino-bearing group.

In another embodiment, the cycloalkylamino-bearing group is selected from the group consisting of a piperazino-bearing 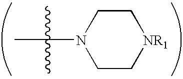
group and a (R1-aminoalkyl)cycloamino-bearing group, wherein R1 equals H, R-amino, acyl, or triazinyl and wherein R1 is selected from the group consisting of a hydrocarbon-containing group, a secondary or tertiary amine-containing group, an oxo-containing group, a thiol-containing group, an amido-containing group, and any combination of one or more of these groups. In yet another embodiment, any one or more of A1-A3 is selected from a group consisting of a sulfur group in a reactive state or an oxygen group in a reactive state. In still another embodiment, the nucleophile-bearing group of A1 and A2 are the same. In a further embodiment, each of the nucleophile-bearing groups of A1 and A2 is different. In still a further embodiment, A1, A2, and A3 are all nucleophile-bearing groups that are the same, while in another embodiment, A1, A2, and A3 are all nucleophile-bearing groups that are different.

In a further embodiment, the unreactive group is selected from the group consisting of a hydrocarbon-containing group, a secondary or tertiary amine-containing group, an oxo-containing group, a thiol-containing group, an amido-containing group, and any combination of one or more of these groups. In another embodiment, one or more of the monomeric electrophilic triazines comprises:

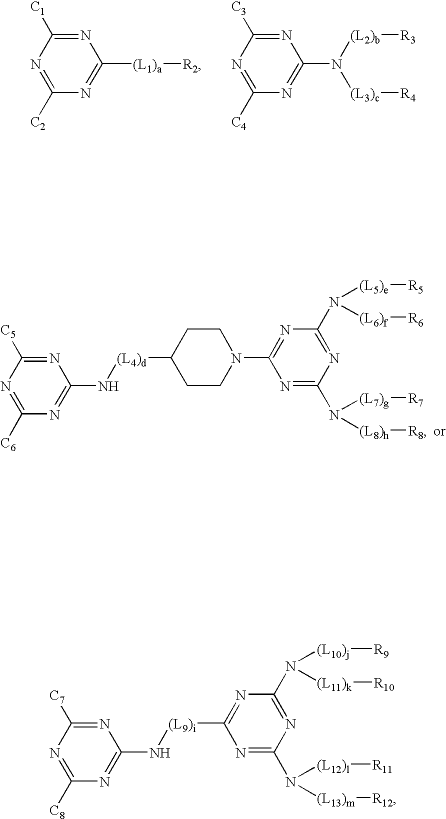

wherein: C1-C8 is each independently a substitutable group, L1-L13 is each independently a linker group, R2-R12 is each independently a protected nucleophilic group, and a-m is each independently 0-200. In yet another embodiment, any one or more of C1-C8 is a halogen. In still another embodiment, the halogen is chlorine. In a further embodiment, any one or more of L1-L13 is selected from the group consisting of a hydrocarbon-containing group, a secondary or tertiary amine-containing group, an oxo-containing group, a thiol-containing group, an amido-containing group, and any combination of one or more of these groups.

In another embodiment, the secondary or tertiary amine-containing group is piperazino- wherein 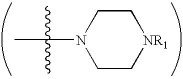
R1 equals H, R-amino, acyl, or triazinyl, and wherein R1 is selected from the group consisting of a hydrocarbon-containing group, a secondary or tertiary amine-containing group, an oxo-containing group, a thiol-containing group, an amido-containing group, and any combination of one or more of these groups. In yet another embodiment, one or more oxo-containing groups is an ether, while in another embodiment the ether is polyethylene glycol. In still another embodiment, any one or more of L1-L13 comprises one or more amido-containing groups, while in another embodiment the one or more amido-containing groups is a protected carbohydrate or a protected peptide. In another embodiment, any one or more of L1-L13 comprises a hydrocarbon group, while in a further embodiment the one or more alkyl groups comprises (—CH2—)a-m. In yet another embodiment, a-g of any one or more of (—CH2—)a-m groups is 2 or 3 (i.e., ethyl or propyl). In still another embodiment, any one or more of R2-R12 is selected from the group consisting of a protected amino group, a protected hydroxyl group, a protected carbonyl group, a protected carboxyl group, a protected thiol group, and a protected phosphate group. In a further embodiment, any one or more of R2-R12 comprises a protected amino group, while in another embodiment the protected amino group is selected from a group consisting of a Boc-protected amino group or an acetyl protected amino group.

In a further embodiment, any one or more -(L1-13)a-m-R2-12) comprises one or more ethylamino groups wherein one or more of the amino groups is protected by a protecting group. In another embodiment, the one or more protecting groups is selected from a group consisting of a Boc group or an acetyl group. In still another embodiment, the protected amino group provides a protecting group that can be readily converted to a nucleophilic amine in one or more steps. In another embodiment, the protecting group that can be readily converted to a nucleophilic amine in one or more steps is selected from a group consisting of a carbamate, an amide, and an imide, while in another embodiment, the carbamate is BOC. In yet another embodiment, the amide is acetyl, while in another embodiment the imide is phthalamidoyl. In another embodiment, any one or more of R2-R12 comprises a protected hydroxyl group. In a further embodiment, a-m is each independently 0-10, while in a further embodiment a-m is each independently 0-2.

Another embodiment of the invention is a method wherein the diversity group is selected from the group consisting of H, a hydrogen-containing group, a carbon-containing group, a secondary or tertiary amine-containing group, an oxo-containing group, a thiol-containing group, an amido-containing group, a phosphino-containing group, a metal-containing group, a nucleophile-bearing group, a protected nucleophile-bearing group, an electrophile-bearing group, a compatibilizing group, a polymer, a resin, a bead, a targeting group, a drug, a solubility enhancer, and any combination of one or more of these groups. In another embodiment, the diversity group is a carbon-containing group attached to the macromolecule, while in yet another embodiment the carbon-containing group is an electrophile-bearing group. In still another embodiment, the carbon-containing group is a nucleophile-bearing group. In yet another embodiment, the diversity group is attached to the macromolecule through an attachment consisting of the group selected from carbon—carbon single bond, an alkene, an amide, a sulfonamide, an ester, an ether, a thioether, a carbonyl, and a thiocarbonyl. In a further embodiment, the diversity group is —(CH2)2-O—(CH2)n-OH or —(CH2)2-O—(CH2)n-OCH3.

In a further embodiment, the diversity group is selected from a group consisting of a protein, a peptide, a carbohydrate, an enzyme, an antibody, an antibacterial agent, an antibiotic, an antiviral agent, an antifungal agent, an anticancer agent, a tumor marker, a cell targeting ligand, a DNA intercalator, an organ-specific ligand, and a compatibilizing group. In yet another embodiment, the compatibilizing group is selected from the group consisting of an anionic group, a cationic group, or a polyalkylene glycol. In still another embodiment, the polyalkylene glycol is polyethylene glycol. In a further embodiment, the diversity group is a targeting group, while in another embodiment the targeting group is a therapeutic agent. In yet another embodiment, the diversity group is attached to the macromolecule via a linker.

In another embodiment, the linker is selected from the group consisting of a hydrocarbon-containing group, a secondary or tertiary amine-containing group, an oxo-containing group, a thiol-containing group, an amido-containing group, a phosphino-containing group, and any combination of one or more of these groups. In yet another embodiment, the linker is selected from the group consisting of

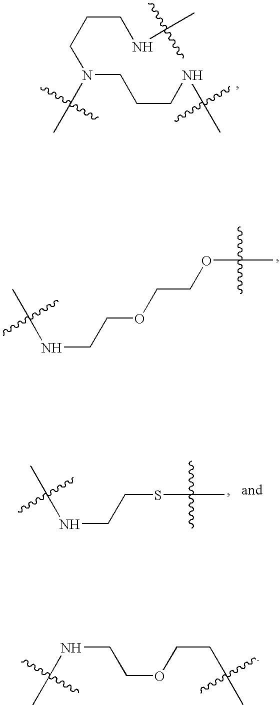

Another embodiment of the invention is a macromolecule synthesized by any one of the methods encompassed by the embodiments described above. In a further embodiment, the macromolecule is selected from the group consisting of:

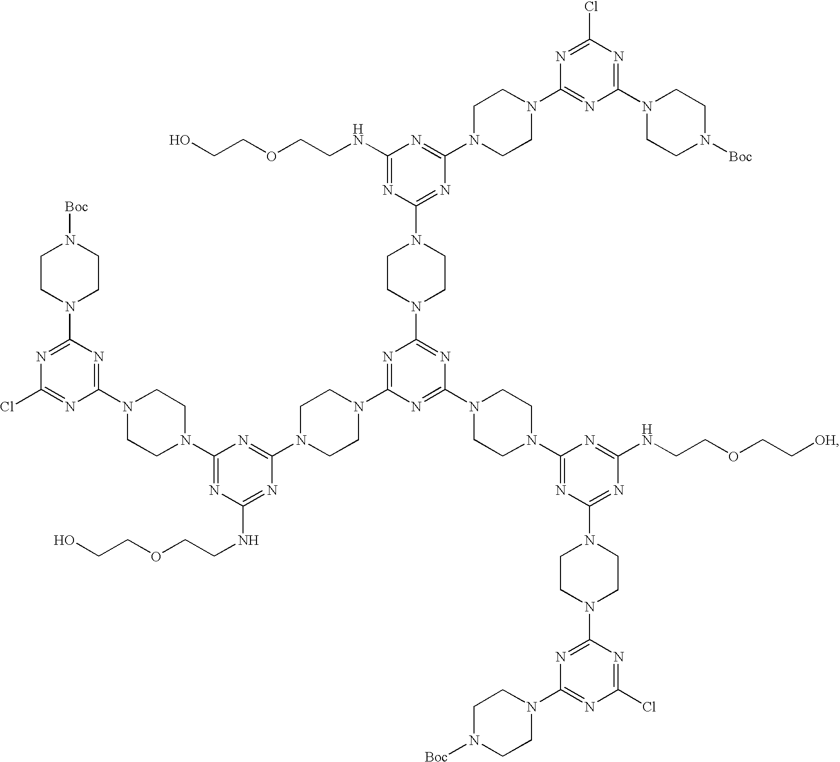

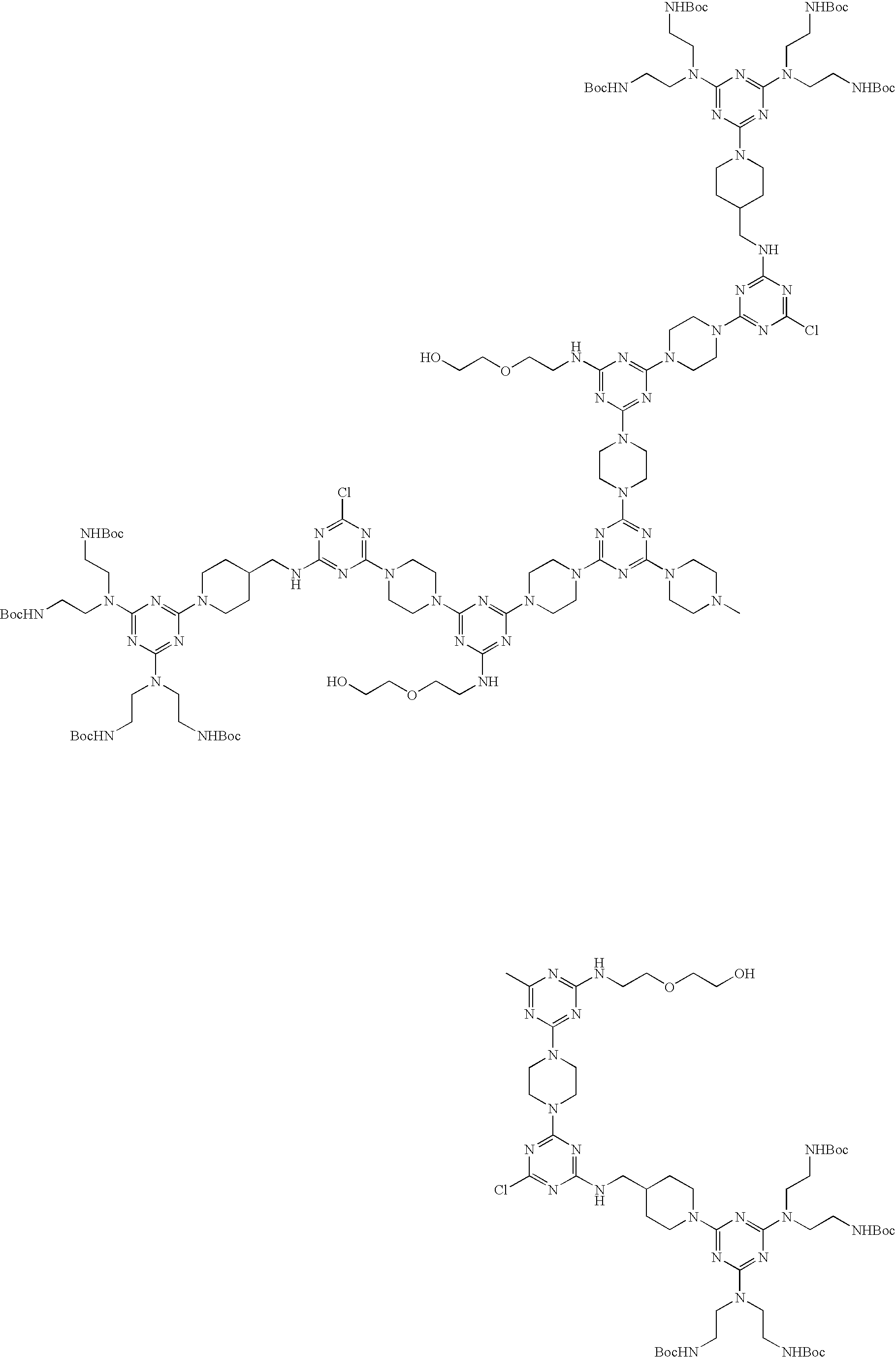

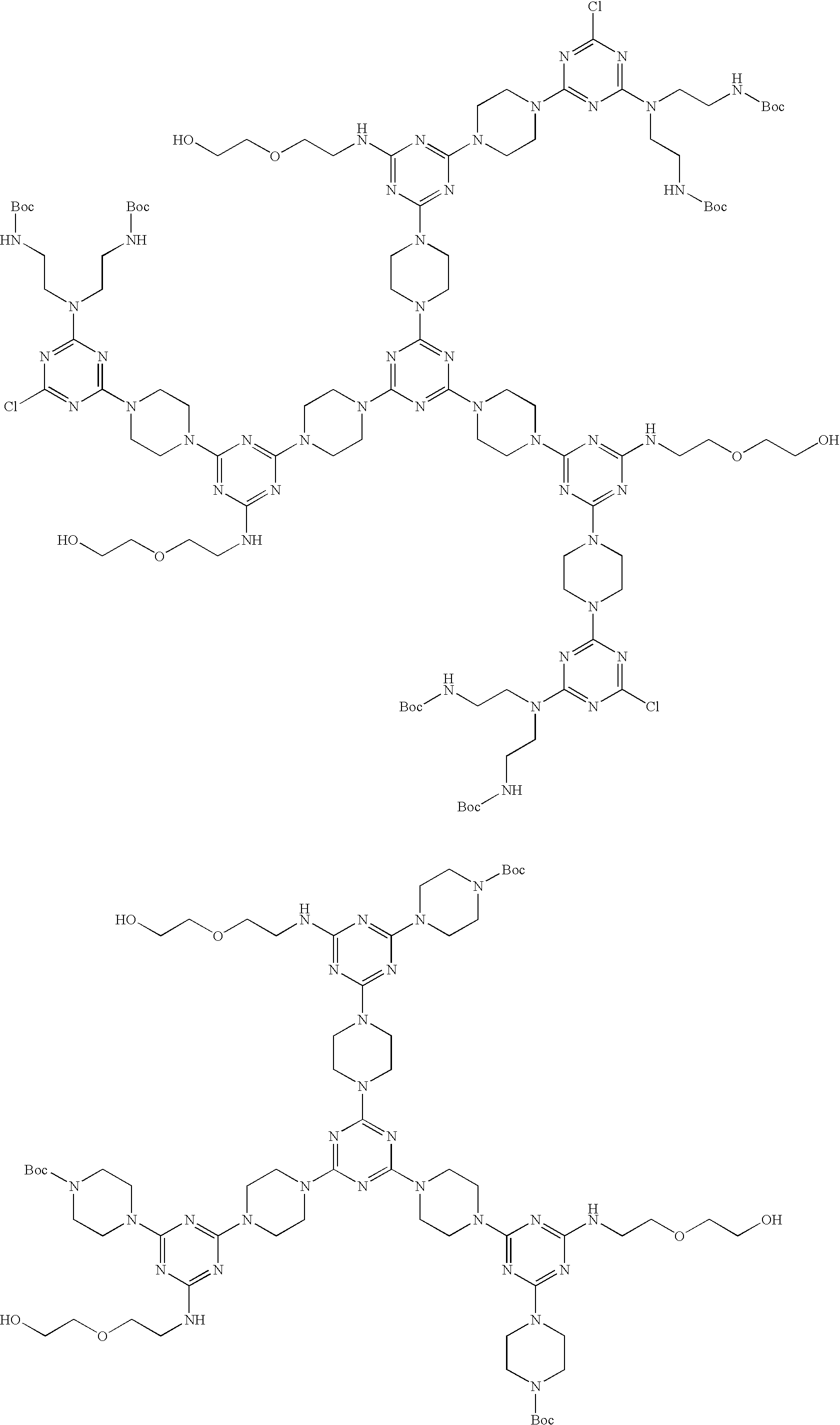

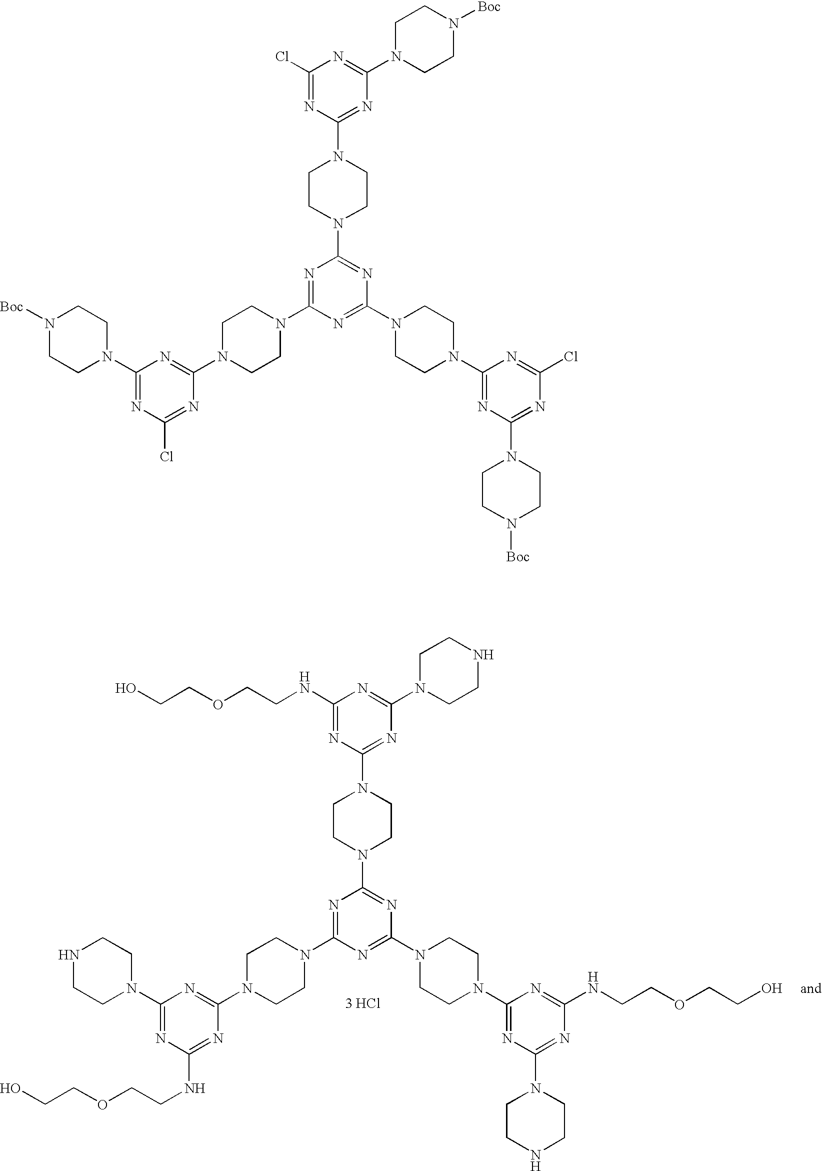

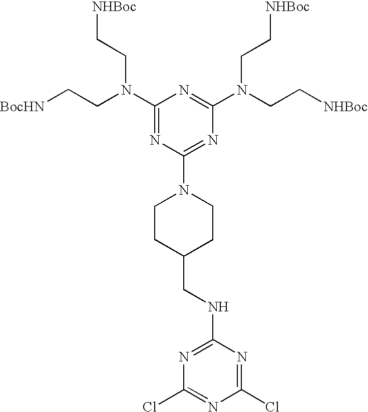

A further embodiment of the invention is a monomeric electrophilic triazine of the formula: 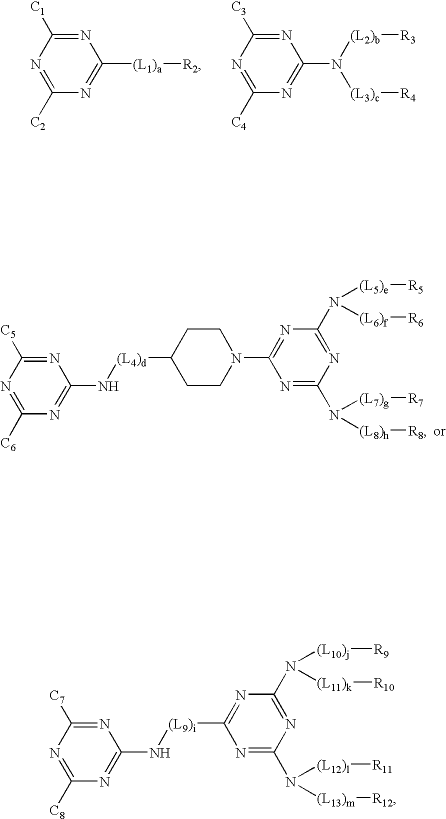
wherein: C1-C8 is each independently a substitutable group, L1-L13 is each independently a linker group, R2-R12 is each independently a protected nucleophilic group, and a-m is each independently 0-200.

Another embodiment of the invention is a compound of the formula: H-J-K, wherein: H comprises a nucleophile-bearing core having one or more nucleophilic groups of the formula: 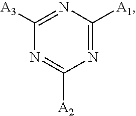
wherein: A1 is a first nucleophile-bearing group, and A2, and A3 are selected from a group consisting of a second nucleophile-bearing group, which may or may not be the same as the first nucleophilic group, a third nucleophilic group, which may or may not be the same as the first and second nucleophilic groups, and an unreactive group; provided that H is bound to J through a nucleophilic reaction wherein one of H's nucleophilic groups has reacted with one of J's electrophilic groups; J comprises a monomeric electrophilic triazine of the formula: 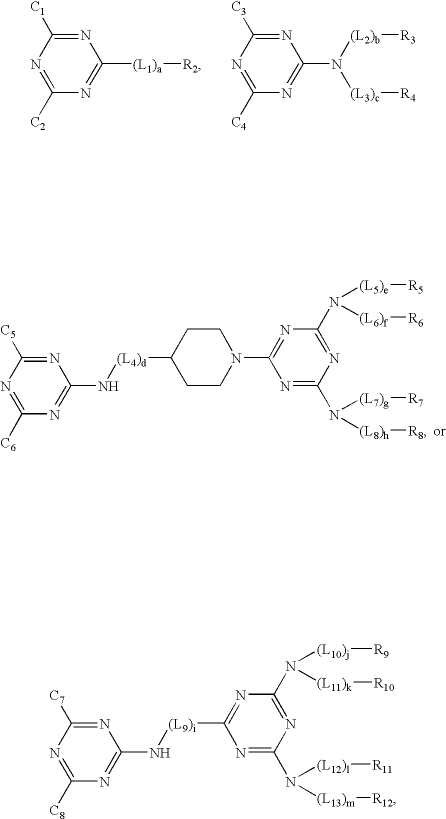
wherein: C1-C8 is each independently a substitutable group, L1-L13 is each independently a linker group, R2-R12 is each independently a protected nucleophilic group, and a-m is each independently 0-200; provided that J is bound to H in the manner described above and one bond of J is bound to K via a covalent bond; and K comprises a diversity group.

One embodiment of the invention is method of treating, diagnosing, or imaging a subject, comprising administering to the subject a pharmaceutically effective amount of a macromolecule prepared by the method of certain embodiments described above. In one embodiment, the subject is selected from the group consisting of a receptor, cell, tissue, organ, or mammal.

Another embodiment of the invention is a method of treating, diagnosing, or imaging a subject, comprising administering to the subject a pharmaceutically effective amount of a macromolecule of the present invention. In one embodiment, the subject is selected from the group consisting of a receptor, cell, tissue, organ, or mammal.

Another embodiment of the invention is a method of treating, diagnosing, or imaging a subject, comprising administering to the subject a pharmaceutically effective amount of a macromolecule of the present invention. In one embodiment, the subject is selected from the group consisting of a receptor, cell, tissue, organ, or mammall

Macromolecules of the invention may contain one or more asymmetric centers and thus can occur as racemates and racemic mixtures, single enantiomers, diastereomeric mixtures and individual diasteromers. All possible stereoisomers of the macromolecules of the present invention are contemplated as being within the scope of the present invention. The chiral centers of the macromolecules of the present invention can have the S— or the R-configuration, as defined by the IUPAC 1974 Recommendations. The present invention is meant to comprehend all such isomeric forms of the compounds of the invention.

The claimed invention is also intended to encompass salts of any of the synthesized macromolecules of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred as described below, although other salts may be useful, as for example in isolation or purification steps.

Examples of acid addition salts include but are not limited to acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pectinate, persulfate, 3-phenylpropionate, picrate, pivalate, propionate, succinate, tartrate, thiocyanate, tosylate, and undecanoate.

Examples of basic salts include but are not limited to ammonium salts; alkali metal salts such as sodium, lithium, and potassium salts; alkaline earth metal salts such as calcium and magnesium salts; salts comprising organic bases such as amines (e.g., dicyclohexylamine, alkylamines such as t-butylamine and t-amylamine, substituted alkylamines, aryl-alkylamines such as benzylamine, dialkylamines, substituted dialkylamines such as N-methyl glucamine (especially N-methyl D-glucamine), trialkylamines, and substituted trialkylamines); and salts comprising amino acids such as arginine, lysine and so forth. The basic nitrogen-containing groups may be quaternized with agents such as lower alkyl halides (e.g. methyl, ethyl. propyl, and butyl chlorides, bromides and iodides), dialkyl sulfates (e.g. dimethyl, diethyl, dibutyl, and diamyl sulfates), long chain halides (e.g. decyl, lauryl, myrtistyl and stearyl chlorides, bromides and iodides), arylalkyl halides (e.g. benzyl and phenethyl bromides), and others known in the art.

Prodrugs and solvates of the macromolecules of the present invention are also contemplated herein. The term “prodrug” as used herein, is understood as being a compound which, upon administration to a subject, such as a mammal, undergoes chemical conversion by metabolic or chemical processes to yield a compound any of the formulas herein, or a salt and/or solvate thereof (Bundgaard, 1991; Bundgaard, 1985). Solvates of the macromolecules of the present invention are preferably hydrates.

The claimed invention is also intended to encompass pharmaceutically acceptable salts of any of the synthesized macromolecules of the present invention. The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium and sodium salts.

Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropyulamine, lysine, methylglucamine, morpholine, piperazine, piperidine, polyamine resins, procaine, purines, theobromine, triethylamine, trimethylamine, tripropylamine, tromethamine, and the like.

When a macromolecule of the present invention is basic, salts may be prepared from pharmaceutically acceptable non-toxic acids, including inorganic and organic acids. Such acids include, but are not limited to, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric and tartaric acids.

As used herein, the term “alkyl,” alone or in combination, refers to a straight-chain or branched-chain alkyl radical containing from 1 to 10, preferably from 1 to 6 and more preferably from 1 to 4, carbon atoms. Examples of such radicals include, but are not limited to, methyl, ethyl, n-propyl, isopropyl, n-butyl, isobutyl, sec-butyl, tert-butyl, pentyl, iso-amyl, hexyl, decyl and the like. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “alkenyl,” alone or in combination, refers to a straight-chain or branched-chain alkenyl radical containing from 2 to 10, preferably from 2 to 6 and more preferably from 2 to 4, carbon atoms having one or more double bonds. Examples of such radicals include, but are not limited to, ethenyl, E- and Z-propenyl, isopropenyl, E- and Z-butenyl, E- and Z-isobutenyl, E- and Z-pentenyl, decenyl and the like. Preferred alkenyl radicals are straight-chain radicals of 3 to 5 carbon atoms and having one double bond. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “alkynyl,” alone or in combination, refers to a straight-chain or branched-chain alkynyl radical containing from 2 to 10, preferably from 2 to 6 and more preferably from 2 to 4, carbon atoms having one or more triple bonds. Examples of such radicals include, but are not limited to, ethynyl (acetylenyl), propynyl, propargyl, butynyl, hexynyl, decynyl and the like. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “cycloalkyl,” alone or in combination, refers to a cyclic alkyl radical containing from 3 to 8, preferably from 3 to 6, carbon atoms. Examples of such cycloalkyl radicals include, but are not limited to, cyclopropyl, cyclobutyl, cyclopentyl, cyclohexyl and the like. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “cycloalkenyl,” alone or in combination, refers to a cyclic carbocycle containing from 4 to 8, preferably 5 or 6, carbon atoms and one or more double bonds. Examples of such cycloalkenyl radicals include, but are not limited to, cyclopentenyl, cyclohexenyl, cyclopentadienyl and the like. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “aryl” refers to a carbocyclic aromatic group selected from the group consisting of phenyl, naphthyl, indenyl, indanyl, azulenyl, fluorenyl, and anthracenyl; or a heterocyclic aromatic group selected from the group consisting of furyl, thienyl, pyridyl, pyrrolyl, oxazolyly, thiazolyl, imidazolyl, pyrazolyl, 2-pyrazolinyl, pyrazolidinyl, isoxazolyl, isothiazolyl, 1,2,3-oxadiazolyl, 1,2,3-triazolyl, 1,3,4-thiadiazolyl, pyridazinyl, pyrimidinyl, pyrazinyl, 1,3,5-triazinyl, 1,3,5-trithianyl, indolizinyl, indolyl, isoindolyl, 3H-indolyl, indolinyl, benzo[b]furanyl, 2,3-dihydrobenzofuranyl, benzo[b]thiophenyl, 1H-indazolyl, benzimidazolyl, benzthiazolyl, purinyl, 4H-quinolizinyl, quinolinyl, isoquinolinyl, innolinyl, phthalazinyl, quinazolinyl, quinoxalinyl, 1,8-naphthyridinyl, pteridinyl carbazolyl, acridinyl, phenazinyl, phenothiazonyl, and phenoxazinyl.

“Aryl” groups, as defined in this application may independently contain one to three substituents which are independently selected from the group consisting of Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino, Ar′-substituted alkyl, Ar′-substituted alkenyl or alkynyl, 1,2-dioxymethylene, 1,2-dioxyethylene, alkoxy, alkenoxy or alkynoxy, Ar′-substituted alkoxy, Ar′-substituted alkenoxy or alkynoxy, alkylamino, alkenylamino or alkynylamino, Ar′-substituted alkylamino, Ar′-substituted alkenylamino or alkynylamino, Ar′-substituted carbonyloxy, alkylcarbonyloxy, aliphatic or aromatic acyl, Ar′-substituted acyl, Ar′-substituted alkylcarbonyloxy, Ar′-substituted carbonylamino, Ar′-substituted amino, Ar′-substituted oxy, Ar′-substituted carbonyl, alkylcarbonylamino, Ar′-substituted alkylcarbonylamino, alkoxy-carbonylamino, Ar′-substituted alkoxycarbonyl-amino, Ar′-oxycarbonylamino, alkylsulfonylamino, mono- or bis-(Ar′-sulfonyl)amino, Ar′-substituted alkyl-sulfonylamino, morpholinocarbonylamino, thiomorpholinocarbonylamino, N-alkyl guanidino, N—Ar+ guanidino, N—N—(Ar′,alkyl) guanidino, N,N′(Ar′,Ar′)guanidino, N,N-dialkyl guanidino, N,N,N-trialkyl guanidino, N-alkyl urea, N,N-dialkyl urea, N-Ar′ urea, N,N—(Ar′,alkyl) urea and N,N′(Ar′)₂ urea; wherein “Ar′” is a carbocyclic or heterocyclic aryl group as defined above having one to three substituents selected from the group consisting of hydrogen, halogen, hydroxyl, amino, nitro, trifluoromethyl, trifluoromethoxy, alkyl, alkenyl, alkynyl, 1,2-dioxymethylene, 1,2-dioxyethylene, alkoxy, alkenoxy, alkynoxy, alkylamino, alkenylamino or alkynylamino, alkylcarbonyloxy, aliphatic or aromatic acyl, alkylcarbonylamino, alkoxycarbonylamino, alkylsulfonylamino, N-alkyl or N,N-dialkyl urea.

The term “oxy,” alone or in combination, refers to an oxygen-containing radical. Examples of suitale oxy groups are ethers, hydroxyls, carbonyls, and oxocarbonyls.

The term “alkoxy,” alone or in combination, refers to an alkyl ether radical, wherein the term “alkyl” is as defined above. Examples of suitable alkyl ether radicals include, but are not limited to, methoxy, ethoxy, n-propoxy, iso-propoxy, n-butoxy, iso-butoxy, sec-butoxy, tert-butoxy and the like. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “alkenoxy,” alone or in combination, refers to a radical of formula alkenyl-O—, wherein the term “alkenyl” is as defined above provided that the radical is not an enol ether. Examples of suitable alkenoxy radicals include, but are not limited to, allyloxy, E- and Z-3-methyl-2-propenoxy and the like. The term “alkynyloxy”, alone or in combination, refers to a radical of formula alkynyl-O—, wherein the term “alkynyl” is as defined above. Examples of suitable alkynoxy radicals include, but are not limited to, propargyloxy, 2-butynyloxy and the like. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “thioalkoxy,” alone or in combination, refers to a thioether radical of formula alkyl-S—, wherein alkyl is as defined above. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “amino,” alone or in combination, is used interchangeably with “amine” and refers to a primary (e.g., —NH₂), secondary (e.g., alkyl-NH—), tertiary (e.g., (alkyl)₂N—), and quarternary (e.g., (alkyl)₃-N(+)-) amine radicals.

The term “alkylamino,” alone or in combination, refers to a mono- or di-alkyl-substituted amino radical (i.e., a radical of formula alkyl-NH— or (alkyl)₂-N—), wherein the term “alkyl” is as defined above. Examples of suitable alkylamino radicals include, but are not limited to, methylamino, ethylamino, propylamino, isopropylamino, t-butylamino, N,N-diethylamino and the like. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “alkenylamino,” alone or in combination, refers to a radical of formula alkenyl-NH— or (alkenyl)₂N—, wherein the term “alkenyl” is as defined above, provided that the radical is not an enamine. An example of such alkenylamino radicals is the allylamino radical. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “alkynylamino,” alone or in combination, refers to a radical of formula alkynyl-NH— or (alkynyl)₂N—, wherein the term “alkynyl” is as defined above, provided that the radical is not an ynamine. An example of such alkynylamino radicals is the propargyl amino radical. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “aryloxy,” alone or in combination, refers to a radical of formula aryl-O—, wherein aryl is as defined above. Examples of aryloxy radicals include, but are not limited to, phenoxy, naphthoxy, pyridyloxy and the like. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “arylamino,” alone or in combination, refers to a radical of formula aryl-NH—, wherein aryl is as defined above. Examples of arylamino radicals include, but are not limited to, phenylamino (anilido), naphthylamino, 2-, 3- and 4-pyridylamino and the like. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “biaryl,” alone or in combination, refers to a radical of formula aryl-aryl-, wherein the term “aryl” is as defined above. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “amido,” alone or in combination, refers to a radical of formula —C(═O)—NH—. Examples of suitable amido-containing radicals include, but are not limited to, amino acids, peptides, proteins and the like.

The term “aryl-fused cycloalkyl,” alone or in combination, refers to a cycloalkyl radical which shares two adjacent atoms with an aryl radical, wherein the terms “cycloalkyl” and “aryl” are as defined above. An example of an aryl-fused cycloalkyl radical is the benzo-fused cyclobutyl radical. Such radicals may be substituted with groups other than hydrogen, such as alkenyl, alkynyl, aryl, amino, halogen, cyano, thio (e.g., thioether, sulfhydryl), oxy (e.g., ether, hydroxy), alkoxy, carboxy, oxocarboxy, and phosphino.

The term “halogen” or “halo” means fluorine, chlorine, bromine or iodine.

The term “leaving group” generally refers to groups readily displaceable by a nucleophile, such as an amine, and alcohol or a thiol nucleophile. Such leaving groups are well known and include carboxylates, N-hydroxysuccinimide, N-hydroxybenzotriazole, halogen (halides), triflates, tosylates, mesylates, alkoxy, thloalkoxy and the like.

The term “functional group” generally refers to how persons of skill in the art classify chemically reactive groups. Examples of functional groups include hydroxyl, amine, sulfhydryl, amide, carboxyls, carbonyls, etc.

The term “triazine” refers to a heteroaryl containing three nitrogens. Such triazines are well known and include 1,3,5-triazine, 1,2,4-triazine, 1,2,3-triazine, and 1,3,4-triazine.

The term “unreactive group” generally refers to a group that does not interfere with or impede the desired transformation of any subsequent reaction.

The term “diversity group” generally refers to any carbon-containing or non-carbon containing group that may be attached to a macromolecule. Examples of diversity groups include protein, a peptide, a carbohydrate, an enzyme, an antibody, an antibacterial agent, an antibiotic, an antiviral agent, an antifungal agent, an anticancer agent, a tumor marker, a cell targeting ligand, a DNA intercalator, an organ-specific ligand, a compatibilizing group and the like.

A convergent synthesis starts with surface groups and through iterative reactions, arrives at larger species that are appended to a central core.

A divergent synthesis starts with a core and does an increasing number of reactions until some (usually large) number of surface grousp are appended.

The term “generation” generally refers to the formation of a macromolecule, such as a dendrimer. Dendrimer, from the Greek word (dendron) for tree, refers to a synthetic, three-dimensional molecule with branching parts. Dendrimers are formed using a nano-scale, multistep fabrication process. Each step results in a new “generation” that has twice the complexity of the previous generation—a first generation dendrimer is the simplest; a tenth generation dendrimer is the most complex and can take months to engineer. Certain dendrimers are called star, dendritic and hybrid macromolecules. Thus, any one macromolecule may consist of one or more generations.

The term “nucleophile” or “nucleophilic” generally refers to atoms bearing lone pairs of electrons. Such terms are well known in the art and include —NH₂, thiolate, carbanion, and alcoholate (also known as hydroxyl).

The term “nucleophile-bearing core” refers to a core group which bears one or more nucleophilic groups. Each nucleophilic group may be protected a protecting group.

The term “electrophilic” and generally refers to species that react with nucleophiles. Electrophilic groups typically have a partial postive charge. Such a term is well known in the art and includes the carbon of a carbon bonded to a leaving group such as a halogen or a quarternary amino group.

The term “attached” generally refers to one compound being bound to another. Such a term is well known in the art and includes two compounds being covalently bound via, for example, an amide or an ester linkage. Non-covalently bound compounds can also be “attached,” such as when a metal is chelated to another compound.

The term “reactive state” refers to a functional group that is prone to participating in a reaction, wherein that participation preferably comprises initiating the reaction.

The term “compatibilizing group” generally refers to a group that conveys a specific property to the macromolecule including but not limited to increasing its solubility in water and/or rendering the macromolecule less antigenic and/or affecting the biodistribution.

The term “iterative steps” generally refers to repetitious cycling of a series of reactions used in general to increase size (generation) or complexity of a molecule; such series of reactions are not necessarily identical in nature or reactants but follow a general discernible theme.

As used throughout this application, the term “mammals” compises humans. And the term “cell” refers to mammalian cells, including human cells.

In view of the above definitions, other chemical terms used throughout this application can be easily understood by those of skill in the art. Terms may be used alone or in any combination thereof. The preferred and more preferred chain lengths of the radicals apply to all such combinations.

Other features or advantages of the present invention will be apparent from the following detailed description of several embodiments, and also from the appending claims.

It is contemplated that any embodiment discussed in this specification can be implemented with respect to any method or composition of the invention, and vice versa. Furthermore, compositions of the invention can be used to achieve methods of the invention.

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The terms “interference,” “inhibiting,” “reducing,” or “prevention,” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims and/or the specification may mean “one,” but it is also consistent with the meaning of “one or more,” “at least one,” and “one or more than one.”

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the examples, while indicating specific embodiments of the invention, are given by way of illustration only. Additionally, it is contemplated that changes and modifications within the spirit and scope of the invention will become apparent to those skilled in the art from this detailed description.

### DESCRIPTION OF ILLUSTRATIVE EMBODIMENTS

The present invention relates generally to synthetic routes for obtaining functional group diversity in macromolecules and to functionally diverse macromolecules. This diversity permits the facile introduction of a variety of diversity groups at multiple positions of these macromolecules, thereby setting the stage for further generational growth of the macromolecule and/or incorporation of other groups such as biocompatible targeting groups. The inventors have identified means of providing facile divergent synthetic methods for the preparation of functionally diverse macromolecules as single-molecule chemical entities. In certain non-limiting aspects, the macromolecules display a variety of diversity groups at multiple positions, thereby setting the stage for further generational growth of the macromolecule and/or incorporation of other diversity groups such as biocompatible targeting groups.

Many macromolecules known in the art bear two or fewer diversity groups. Macromolecules featuring three or more diversity groups are more attractive as polymeric therapeutics, as more sites are available for conjugation of biocompatible groups, reporter groups to indicate biodistribution, and pharmacologically active molecules.

For example, if a macromolecule features a diversity group that is incompatible with a particular environment, the availability of diversifying other portions of the macromolecule to encourage compatibility with the particular environment is a useful application of these macromolecules.

Other uses for macromolecules of the present invention include molecular recognition, supramolecular self-assembly, decoration of inorganic groups, energy harvesting and emitting applications, srufactants and medicinal applications.

Macromolecules can be prepared via convergent or divergent syntheses. A convergent synthesis starts with surface groups and through iterative reactions, arrives at larger species that are appended to a central core. A divergent synthesis starts with a core and does an increasing number of reactions until some (usually large) number of surface groups are appended. The methods of the present invention relate to divergent syntheses.

Divergent syntheses of macromolecules as shown by this invention offer an advantage to many convergent syntheses known in the art as well as some divergent syntheses: the macromolecules of the present invention can be obtained as single-chemical entities (ie. high chemical purity). This is an attractive features, especially for biomedical applications.

Generally speaking, macromolecules of the present invention are prepared by treatment of the poly(nucleophilic) core with monomeric electrophilic triazines to cleanly yields poly(monomeric electrophilic triazines). Subsequent nucleophilic reactions with amine nucleophiles bearing a functional group or diversity group of interest yield diversity. If a substituent on any one monomeric electrophilic triazines is a protected nucleophile, removal of the functional group allows for iterative reactions and the synthesis of star, dendritic and hybrid macromolecules.

The syntheses of these compounds proceeds efficiently and with effective chromatography to produce single-chemical species. A nucleophilic core such as a triazine-based core can be reacted in the presence of mild bases such as diisopropylethylamine in the presence of monomeric electrophilic triazines at low temperatures (0 degrees C.) to produce first generation macromolecules. Depending on the functionality of the starting triazines, such a macromolecule can bear nucleophilic groups, protected nucleophilic groups as well as diversity groups as described herein. Further iterative reactions of the synthesized molecule can result in larger macromolecules also bearing nucleophilic groups, protected nucleophilic groups as well as diversity groups as described herein. A benefit to such “generational expansion” of the present synthesis lies in the fact that the generated macromolecules are pure and available for further reactions.

Not only can a variety of nucleophilic, protected nucleophilic and diversity groups be appended to these macromolecules, but linkers of various lengths can connect one group to the core structure (e.g., one nucleophile-bearing group to a nucleophilic core; a diversity group to a nucleophilic core). This allows for even further extension of the “reach” of each “arm” of any macromolecule of the present invention.

The following information provides additional information regarding various aspects of the macromolecules of the present invention.

A. Protecting Groups

When a chemical reaction is to be carried out selectively at one reactive site in a multifunctional compound, other reactive sites must be temporarily blocked. A “protecting group,” or “protected-nucleophilic group” as used herein, is defined as a group used for the purpose of this temporary blockage. During the synthesis of the macromolecules of the present invention, various functional groups must be protected using protecting groups (or protecting agents) at various stages of the synthesis. For example, various positions of a monomeric electrophilic triazine may need to be protected when used in a synthetic procedure (e.g, “a first monomeric electrohilic triazine having one or more protected nucleophilic groups”). When a diversity group is introduced during the synthesis of a macromolecule, that diversity group may also need to be protected by protecting groups (i.e., a “protected diversity group”). However, use of the phrase or “protected diversity group” or “protected macromolecule” does not mean that every functional group available to be protected is protected. Functional groups necessary for the desired transformation should be unprotected.

There are a number of methods well known to those skilled in the art for accomplishing such a step. For protecting agents, their reactivity, installation and use, see, e.g., “Protective Groups in Organic Synthesis”, 3^{rd} ed., by T. W. Greene and P. G. M. Wuts, John Wiley & Sons, New York, N.Y. (1999), herein incorporated by reference in its entirety. The function of protecting group is to protect one or more functionalities (e.g., —NH₂, —SH, —COOH) of during subsequent reactions which would not proceed well, either because the free (in other words, unprotected) functional group would react and be functionalized in a way that is inconsistent with its need to be free for subsequent reactions, or the free functional group would interfere in the reaction. The same protecting group may be used to protect one or more of the same or different functional group(s).

When a protecting group is no longer needed, it is removed by methods well known to those skilled in the art. For deprotecting agents and their use, see, e.g., “Protective Groups in Organic Synthesis”, 3^{rd} ed., by T. W. Greene and P. G. M. Wuts, John Wiley & Sons, New York, N.Y. (1999). Agents used to remove the protecting group are called deprotecting agents. Protecting groups must be readily removable (as is known to those skilled in the art) by methods employing deprotecting agents that are well known to those skilled in the art. It is well known that certain deprotecting agents remove some protective groups and not others, while other deprotecting agents remove several types of protecting groups from several types of functional groups. Thus, a first deprotecting agent may be used to remove one type of protecting group, followed by the use of a second deprotecting agent to remove a second type of protecting group, and so on.

In one embodiment of the present invention, the deprotecting agent is the use of concentrated HCl in methanol in order to remove a t-butoxycarbonyl group to reveal a free amine (—NH₂). Persons of ordinary skill in the art will be familiar with the proper ordering of protective group removal using deprotecting agents. See e.g., “Protective Groups in Organic Synthesis”, 3^{rd} ed., by T. W. Greene and P. G. M. Wuts, John Wiley & Sons, New York, N.Y. (1999). Particular non-limited examples of protecting groups are discussed below.

1. Amino Protecting Groups

Amino protecting groups are well known to those skilled in the art. See, for example, “Protective Groups in Organic Synthesis”, 3^{rd} ed., by T. W. Greene and P. G. M. Wuts, John Wiley & Sons, New York, N.Y. (1999) Chapter 7.

A suitable amino protecting group may be selected from the group consisting of t-butoxycarbonyl, benzyloxycarbonyl, formyl, trityl, acetyl, trichloroacetyl, dichloroacetyl, chloroacetyl, trifluoroacetyl, difluoroacetyl, fluoroacetyl, benzyl chloroformate, 4-phenylbenzyloxycarbonyl, 2-methylbenzyloxycarbonyl, 4-ethoxybenzyloxycarbonyl, 4-fluorobenzyloxycarbonyl, 4-chlorobenzyloxycarbonyl, 3-chlorobenzyloxycarbonyl, 2-chlorobenzyloxycarbonyl, 2,4-dichlorobenzyloxycarbonyl, 4-bromobenzyloxycarbonyl, 3-bromobenzyloxycarbonyl, 4-nitrobenzyloxycarbonyl, 4-cyanobenzyloxycarbonyl, 2-(4-xenyl)isopropoxycarbonyl, 1,1-diphenyleth-1-yloxycarbonyl, 1,1-diphenylprop-1-yloxycarbonyl, 2-phenylprop-2-yloxycarbonyl, 2-(p-toluyl)prop-2-yloxycarbonyl, cyclopentanyloxycarbonyl, 1-methylcyclopentanyloxycarbonyl, cyclohexanyloxycarbonyl, 1-methylcyclohexanyloxycabonyl, 2-methylcyclohexanyloxycarbonyl, 2-(4-toluylsulfonyl)ethoxycarbonyl, 2-(methylsulfonyl)ethoxycarbonyl, 2-(triphenylphosphino)ethoxycarbonyl, fluorenylmethoxycarbonyl, 2-(trimethylsilyl)ethoxycarbonyl, allyloxycarbonyl, 1-(trimethylsilylmethyl)prop-1-enyloxycarbonyl, 5-benzisoxalylmethoxycarbonyl, 4-acetoxybenzyloxycarbonyl, 2,2,2-trichloroethoxycarbonyl, 2-ethynyl-2-propoxycarbonyl, cyclopropylmethoxycarbonyl, 4-(decyloxyl)benzyloxycarbonyl, isobomyloxycarbonyl, 1-piperidyloxycarbonyl and 9-fluorenylmethyl carbonate.

2. Thiol Protecting Groups

Thiol protecting groups are well known to those skilled in the art. See, for example, “Protective Groups in Organic Synthesis”, 3^{rd} ed., by T. W. Greene and P. G. M. Wuts, John Wiley & Sons, New York, N.Y. (1999) Chapter 6.

A suitable thiol protecting group may be selected from the group consisting of acetamidomethyl, benzamidomethyl, 1-ethoxyethyl, benzoyl, triphenylmethyl, t-butyl, benzyl, adamantyl, cyanoethyl, acetyl, and trifluoroacetyl.

3. Alcohol Protecting Groups

Alcohol protecting groups are well known to those skilled in the art. See, for example, “Protective Groups in Organic Synthesis”, 3^{rd} ed., by T. W. Greene and P. G. M. Wuts, John Wiley & Sons, New York, N.Y. (1999) Chapter 6.

A suitable alcohol protecting group may be selected from the group consisting of methoxymethyl, (phenyldimethylsilyl)methoxymethyl, beynzyloxymethyl, t-butoxymethyl, and tetrahydropyranyl.

5. Carbonyl Protecting Groups

Carbonyl protecting groups are well known to those skilled in the art. See, for example, “Protective Groups in Organic Synthesis”, 3^{rd} ed., by T. W. Greene and P. G. M. Wuts, John Wiley & Sons, New York, N.Y. (1999) Chapter 6.

A suitable carboxylic acid protecting group may be selected from the group consisting of dimethylacetal, dimethylketal, diisopropylacetal, diisopropylketal, enamines and enol ethers.

4. Carboxylic Acid Protecting Groups

Carboxylic acid protecting groups are well known to those skilled in the art. See, for example, “Protective Groups in Organic Synthesis”, 3^{rd} ed., by T. W. Greene and P. G. M. Wuts, John Wiley & Sons, New York, N.Y. (1999) Chapter 6.

A suitable carboxylic acid protecting group may be selected from the group consisting of dimethylacetal, methoxymethylester, phenylacetoxymethyl ester and tetrahydropyranyl ester.

B. Source of Compounds, Agents, and Active Ingredients

The compounds, agents, and active ingredients (e.g., solvents, catalysts, bases used in reactions, and other compounds, agents, and active ingredients described herein) that are described in the claims and specification can be obtained by any means known to a person of ordinary skill in the art. In a non-limiting embodiment, for example, the compounds, agents, and active ingredients can be isolated by obtaining the source of such compounds, agents, and active ingredients. In many instances, the compounds, agents, and active ingredients are commercially available. See, e.g., sigmaaldrich.com.

C. Modifications and Derivatives

Modifications or derivatives of the compounds, agents, and active ingredients disclosed throughout this specification are contemplated as being useful with the methods and compositions of the present invention. Derivatives may be prepared and the properties of such derivatives may be assayed for their desired properties by any method known to those of skill in the art.

In certain aspects, “derivative” refers to a chemically modified compound that still retains the desired effects of the compound prior to the chemical modification. Such derivatives may have the addition, removal, or substitution of one or more chemical moieties on the parent molecule. Non limiting examples of the types modifications that can be made to the compounds and structures disclosed throughout this document include the addition or removal of lower alkanes such as methyl, ethyl, propyl, or substituted lower alkanes such as hydroxymethyl or aminomethyl groups; carboxyl groups and carbonyl groups; hydroxyls; nitro, amino, amide, and azo groups; sulfate, sulfonate, sulfono, sulfhydryl, sulfonyl, sulfoxido, phosphate, phosphono, phosphoryl groups, and halide substituents. Additional modifications can include an addition or a deletion of one or more atoms of the atomic framework, for example, substitution of an ethyl by a propyl; substitution of a phenyl by a larger or smaller aromatic group. Alternatively, in a cyclic or bicyclic structure, hetero atoms such as N, S, or O can be substituted into the structure instead of a carbon atom.

D. Equivalents

Known and unknown equivalents to the specific compounds, agents, and active ingredients discussed throughout this specification can be used with the compositions and methods of the present invention. The equivalents can be used as substitutes for the specific compounds, agents, and active components. The equivalents can also be used to add to the methods and compositions of the present invention. A person of ordinary skill in the art would be able to recognize and identify acceptable known and unknown equivalents to the specific compounds, agents, and active ingredients without undue experimentation.

E. Compositions of the Present Invention

A person of ordinary skill would recognize that the that the compounds of the present invention may be formulated into useful compositions, in particular, pharmaceutical (diagnostic or therapeutic) compositions, as such compounds may be modified to include any number of combinations of compounds, agents, and/or active ingredients, or derivatives therein. It is also contemplated that that the concentrations of the compounds, agents, and/or active ingredients can vary. In non-limiting embodiments, for example, the compositions may include in their final form, for example, at least about 0.0001% to 99% or any range derivable therein, of at least one of the compounds, agents, active ingredients, or derivatives. In non-limiting aspects, the percentage can be calculated by weight or volume of the total composition. A person of ordinary skill in the art would understand that the concentrations can vary depending on the addition, substitution, and/or subtraction of the compounds, agents, or active ingredients, to the disclosed methods and compositions.

F. Vehicles

The compositions of the present invention can be incorporated into all types of are effective in all types of vehicles. Non-limiting examples of suitable vehicles include emulsions (e.g., water-in-oil, water-in-oil-in-water, oil-in-water, -oil-in-water-in-oil, oil-in-water-in-silicone emulsions), creams, lotions, solutions (both aqueous and hydro-alcoholic), anhydrous bases (such as lipsticks and powders), gels, and ointments or by other method or any combination of the forgoing as would be known to one of ordinary skill in the art (Remington's, 1990). Variations and other appropriate vehicles will be apparent to the skilled artisan and are appropriate for use in the present invention. In certain aspects, it is important that the concentrations and combinations of the compounds, ingredients, and active agents be selected in such a way that the combinations are chemically compatible and do not form complexes which precipitate from the finished product.

G. Cosmetic Products and Articles of Manufacture

The composition of the present invention can also be used in many cosmetic products including, but not limited to, sunscreen products, sunless skin tanning products, hair products, finger nail products, moisturizing creams, skin benefit creams and lotions, softeners, day lotions, gels, ointments, foundations, night creams, lipsticks, cleansers, toners, masks, or other known cosmetic products or applications. Additionally, the cosmetic products can be formulated as leave-on or rinse-off products.

H. Targeting Ligands

In some embodiments of the compositions of the present invention, a targeting ligand, or more particularly tissue-specific ligands, may be attached to the macromolecule compound. A “tissue-specific ligand” or “tissue-targeting ligand” is defined herein to refer to a part of a molecule that can bind or attach to one or more tissues. The binding may be by any mechanism of binding known to those of ordinary skill in the art (e.g., covalent, non-covalent, associative). Examples include therapeutic agents, antimetabolites, apoptotic agents, bioreductive agents, signal transductive therapeutic agents, receptor responsive agents, or cell cycle specific agents. The tissue may be any type of tissue, such as a cell. For example, the cell may be the cell of a subject, such as a cancer cell. In certain embodiments, the tissue targeting moiety is a tissue targeting amino acid sequence that is chemically conjugated or fused to a macromolecule that is capable of binding to a valent metal ion.

Examples of targeting ligands include disease cell cycle targeting compounds, tumor angiogenesis targeting ligands, tumor apoptosis targeting ligands, disease receptor targeting ligands, drug-based ligands, antimicrobials, tumor hypoxia targeting ligands, an agent that mimics glucose, amifostine, angiostatin, EGF receptor ligands, capecitabine, COX-2 inhibitors, deoxycytidine, fullerene, herceptin, human serum albumin, lactose, leuteinizing hormone, pyridoxal, quinazoline, thalidomide, transferrin, and trimethyl lysine.

In further embodiments of the present invention, the tissue-specific moiety is an antibody. Any antibody is contemplated as a tissue-targeting moiety in the context of the present invention. For example, the antibody may be a monoclonal antibody. One of ordinary skill in the art would be familiar with monoclonal antibodies, methods of preparation of monoclonal antibodies, and methods of use of monoclonal antibodies as ligands. In certain embodiments of the present invention, the monoclonal antibody is an antibody directed against a tumor marker. In some embodiments, the monoclonal antibody is monoclonal antibody C225, monoclonal antibody CD31, or monoclonal antibody CD40.

I. Therapeutic Agents

In certain embodiments of the compositions of the present invention, a targeting or tissue-specific ligand is bound to the macromolecule. A “moiety” is defined herein to be a part of a molecule. In certain particular embodiments, the tissue-specific ligand is a therapeutic moiety. A “therapeutic moiety” is defined herein to refer to any therapeutic agent. A “therapeutic agent” is defined herein to include any compound or substance or drug that can be administered to a subject, or contacted with a cell or tissue, for the purpose of treating a disease or disorder, or preventing a disease or disorder, or treating or preventing an alteration or disruption of a normal physiologic process. For example, the therapeutic moiety may be an anti-cancer moiety, such as a chemotherapeutic agent. In certain embodiments of the present invention, the therapeutic moiety is a therapeutic amino acid sequence that is fused or chemically conjugated to the therapeutic amino acid sequence. Such chemical conjugates and fusion proteins are discussed further in other parts of this specification.

Examples of anti-cancer moieties include any chemotherapeutic agent known to those of ordinary skill in the art. Examples of such chemotherapeutic agents include, but are not limited to, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing. In certain particular embodiments, the anti-cancer moiety is methotrexate.

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids such as retinoic acid; capecitabine; and pharmaceutically acceptable salts, acids or derivatives of any of the above.

Also included in this definition are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene; aromatase inhibitors that inhibit the enzyme aromatase, which regulates estrogen production in the adrenal glands, such as, for example, 4(5)-imidazoles, aminoglutethimide, megestrol acetate, exemestane, formestanie, fadrozole, vorozole, letrozole, and anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor and a HER2 expression inhibitor; vaccines such as gene therapy vaccines and pharmaceutically acceptable salts, acids or derivatives of any of the above.

A list of FDA-approved oncology drugs with approved indications and date of approval can be found on the world wide web at accessdata.fda.gov/scripts/cder/onctools/druglist.cfm.

J. Imaging Moieties

In certain embodiments of the compositions of the present invention, the tissue-specific ligand is an imaging moiety. As defined herein, an “imaging moiety” is a part of a molecule that is a agent or compound that can be administered to a subject, contacted with a tissue, or applied to a cell for the purpose of facilitating visualization of particular characteristics or aspects of the subject, tissue, or cell through the use of an imaging modality. Imaging modalities are discussed in greater detail below. Any imaging agent known to those of ordinary skill in the art is contemplated as an imaging moiety of the present invention. Thus, for example, in certain embodiments of the compositions of the present invention, the compositions can be applied in multimodality imaging techniques. Dual imaging and multimodality imaging are discussed in greater detail in the specification below.

In certain embodiments, the imaging moiety is a contrast media. Examples include CT contrast media, MRI contrast media, optical contrast media, ultrasound contrast media, or any other contrast media to be used in any other form of imaging modality known to those of ordinary skill in the art. Examples include diatrizoate (a CT contrast agent), a gadolinium chelate (an MRI contrast agent), and sodium fluorescein (an optical contrast media). Additional examples of contrast media are discussed in greater detail in the specification below. One of ordinary skill in the art would be familiar with the wide range of types of imaging agents that can be employed as imaging moieties in the macromolecules of the present invention.

K. Emulsifiers

The compositions of the present invention can also comprise one or more emulsifiers. Emulsifiers can reduce the in interfacial tension between phases and improve the formulation and stability of an emulsion. The emulsifiers can be nonionic, cationic, anionic, and zwitterionic emulsifiers (See McCutcheon's (1986); U.S. Pat. Nos. 5,011,681; 4,421,769; 3,755,560). Non-limiting examples include esters of glycerin, esters of propylene glycol, fatty acid esters of polyethylene glycol, fatty acid esters of polypropylene glycol, esters of sorbitol, esters of sorbitan anhydrides, carboxylic acid copolymers, esters and ethers of glucose, ethoxylated ethers, ethoxylated alcohols, alkyl phosphates, polyoxyethylene fatty ether phosphates, fatty acid amides, acyl lactylates, soaps, TEA stearate, DEA oleth-3 phosphate, polyethylene glycol 20 sorbitan monolaurate (polysorbate 20), polyethylene glycol 5 soya sterol, steareth-2, steareth-20, steareth-21, ceteareth-20, PPG-2 methyl glucose ether distearate, ceteth-10, polysorbate 80, cetyl phosphate, potassium cetyl phosphate, diethanolamine cetyl phosphate, polysorbate 60, glyceryl stearate, PEG-100 stearate, and mixtures thereof.

### EXAMPLES

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

With respect to the following Examples, ¹H and ¹³C{¹H} NMR data were acquired on a Varian 300 MHz spectrometer at RT unless otherwise indicated. ¹H and ¹³C{¹H} NMR chemical shifts are listed relative to tetramethylsilane in parts per million, and were referenced to the residual proton or carbon peak of the solvent. MS analyses were performed by the Laboratory for Biological Mass Spectrometry at Texas A&M University. All solvents and reagents were purchased from Aldrich Chemical Co. or Acros Organics and were used without purification. The reagents 4,ⁱ 7ⁱⁱ and 13ⁱⁱⁱ were prepared by a modification of previously reported methods. Thin-layer chromatography was performed using EMD silica gel 60 F₂₅₄ pre-coated glass plates (0.25 mm), and preparative chromatography was performed using EMD silica gel 60 (0.040 mm particle size).

The following synthetic diagrams, chemical structures and synthetic details provide certain macromolecules of the present invention.

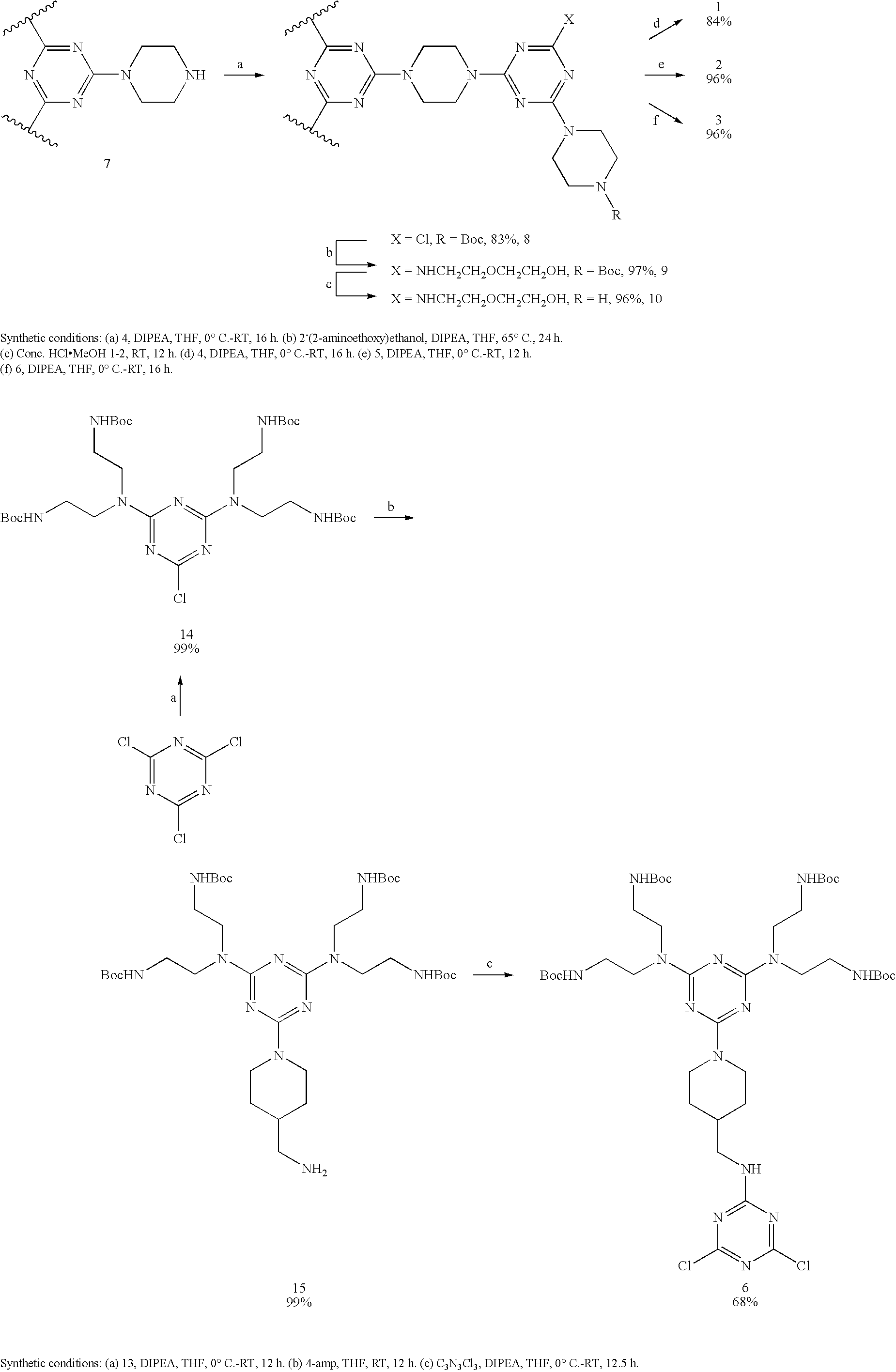

| | | | Overall | Col- |
|---|---|---|---|---|
| Cmpd | Structure | Steps | Yield^{a} | umns |
| 1 | 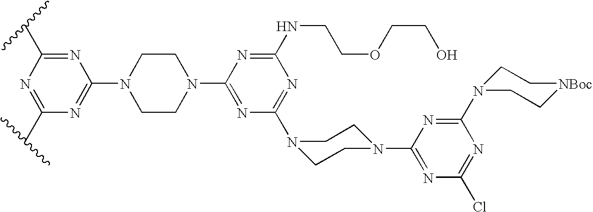 | 4 | 65% | 2 |
| 2 | 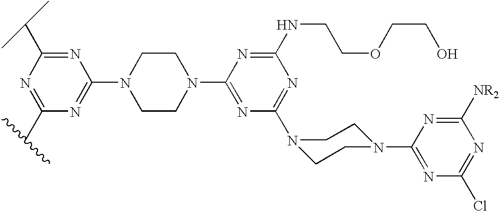 | 4 | 75% | 2 |
| | R = CH₂CH₂NHBoc | | | |
| 3 | 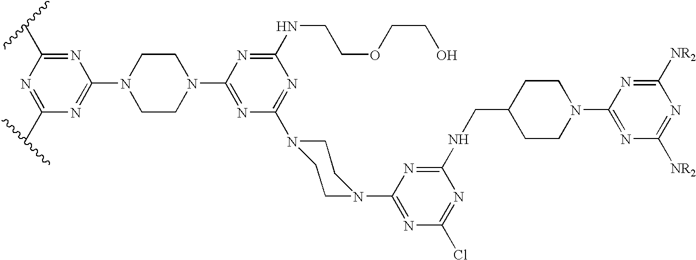 | 4 | 74% | 2 |
| | R = CH₂CH₂NHBoc | | | |

^{a}Precluding contributions from the syntheses of 4-7.

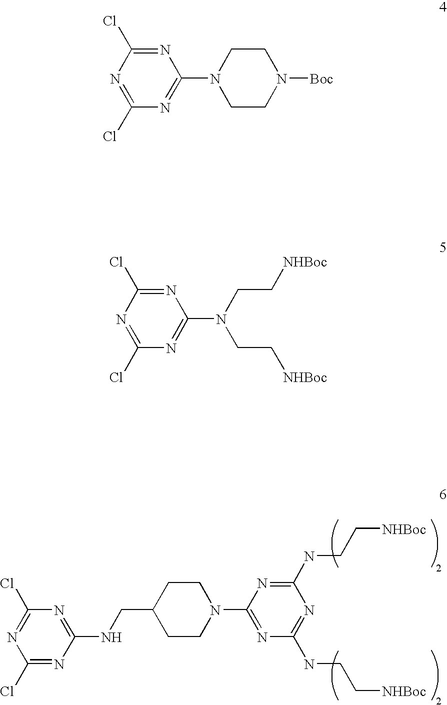

### Example 1

1: A clear solution of 4 (499 mg, 1.49 mmol) in 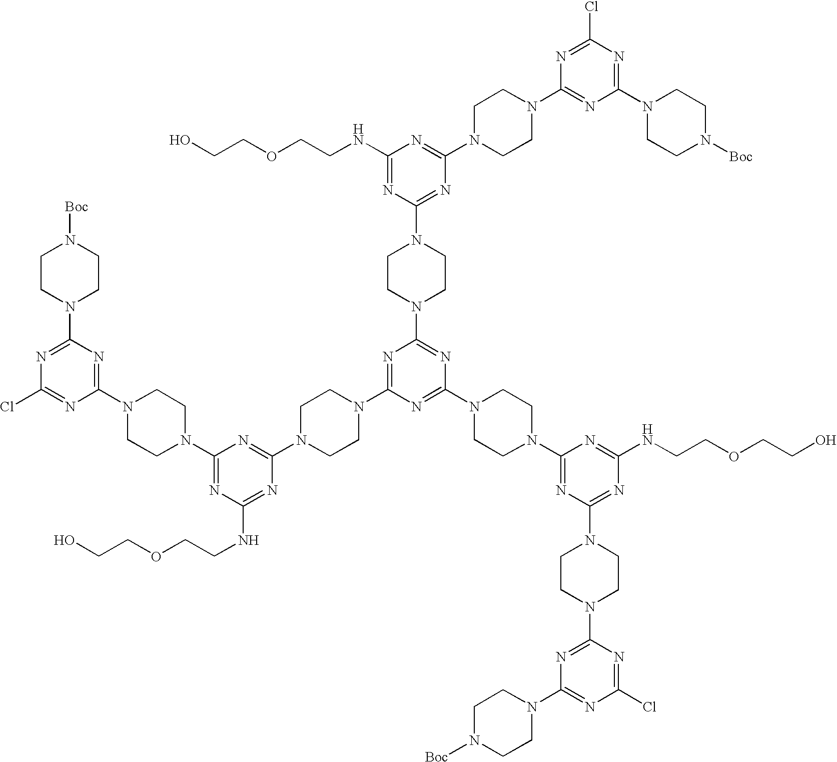
50 mL of THF was prepared, and a slurry of (NH)₃OH₃·3HCl (530 mg, 0.43 mmol) and DIPEA (0.73 mL, 4.29 mmol) was prepared in 50 mL of THF in a separate flask. Both solutions were cooled to 0° C., then combined. The mixture was warmed gradually to RT and stirred for 16 h, after which time the solvent was removed in vacuo. Purification was achieved using column chromatography on silica gel to afford the product as a white solid (gradient elution from 80:1 CH₂Cl₂:MeOH until no detectable 4 was observed as determined using UV spotting to 20:1 CH₂Cl₂:MeOH to obtain the desired product; R_{f}=0.24 using 9:1 CH₂Cl₂:MeOH as the developing solvent). Yield: 726 mg (84%). The excess/unreacted C₃N₃(pip-Boc)Cl₂ was also recovered from this purification (R_{f}=0.90 using 9:1 CH₂Cl₂:MeOH as the developing solvent). Yield: 139 mg (99% of unreacted starting material based on recovered yield of star polymer). ¹H NMR (300 MHz, CDCl₃) □: 5.44 (br, 3H, NH), 3.77 (br, 60H+6H, CH₂), 3.61 (br, 18H, CH₂), 3.44 (br, 12H, CH₂NBoc), 2.30 (br, 3H, OH), 1.45 (s, 27H, C(CH₃)₃). ¹³C{¹H} NMR (75.5 MHz, CDCl₃) □: 169.6 (s, C₃N₃), 165.3 (s, C₃N₃), 164.4 (s, C₃N₃), 154.6 (s, C(O)), 80.2 (s, C(CH₃)₃), 72.4 (s, OCH₂CH₂OH), 70.0 (s, NHCH₂CH₂O), 61.6 (s, OCH₂CH₂OH), 43.2 and 43.0 (s, CH₂CH₂NHBoc, piperazine), 40.5 (s, NHCH₂CH₂O), 28.3 (s, C(CH₃)₃). MS (MALDI): calcd 2022.9841 (M⁺); found 2024.0846 (M+H⁺).

### Example 2

2: A clear solution of 5 (177 mg, 0.39 mmol) in 10 mL 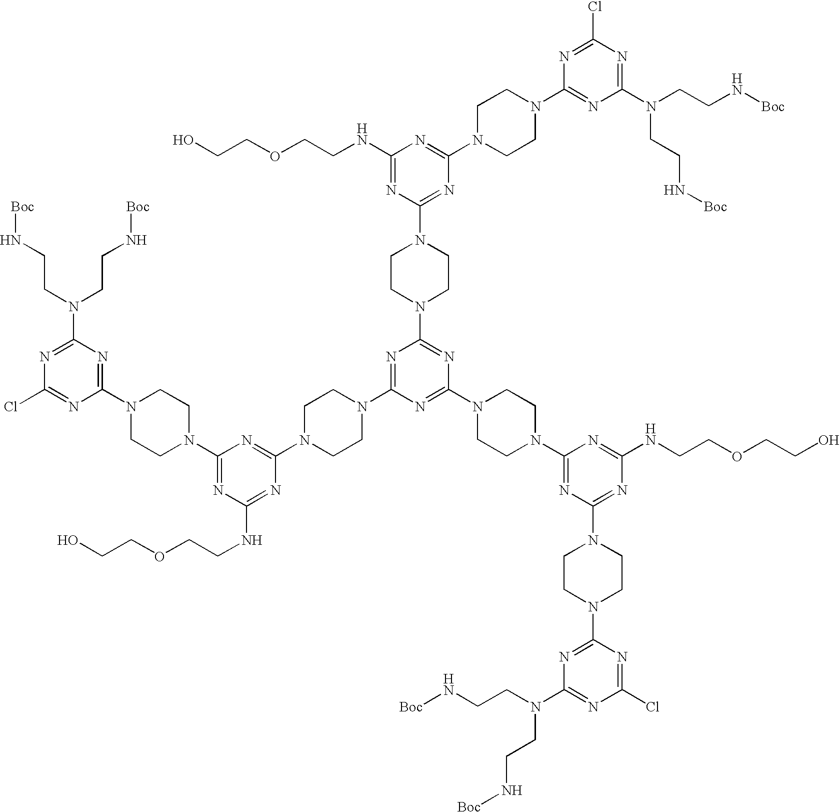
of THF was prepared, and a slurry of 10 (122 mg, 9.8×10⁻⁵ mol) and DIPEA (0.17 mL, 1.00 mmol) was prepared in 20 mL of THF in a separate flask. Both solutions were cooled to 0° C., then combined. The mixture was warmed gradually to RT and stirred for 16 h, after which time the solvent was removed in vacuo. Purification was achieved using column chromatography on silica gel to afford the product as a white solid (gradient elution from 80:1 CH₂Cl₂:MeOH until no detectable 5 was observed as determined using UV spotting to 20:1 CH₂Cl₂:MeOH to obtain the desired product; R_{f}=0.54 using 9:1 CH₂Cl₂: MeOH as the developing solvent). Yield: 227 mg (97%). The excess/unreacted 5 was also recovered from this purification (R_{f}=0.76 using 9:1 CH₂Cl₂:MeOH as the developing solvent). Yield: 48 mg (99% of unreacted starting material based on recovered yield of hybrid polymer). ¹H NMR (300 MHz, CDCl₃) □: 5.25 (br, 3H, NH), 5.17 (br, 3H, NH), 5.09 (br, 3H, NH), 3.76 (br, 54H, CH₂), 3.62 (m, 30H, CH₂), 3.35 (br, 12H, CH₂NHBoc), 2.20 (br, 3H, OH), 1.40 (s, 27H, C(CH₃)₃), 1.37 (s, 27H, C(CH₃)₃). ¹³C{¹H} NMR (75.5 MHz, CDCl₃) □: 169.4 (s, C₃N₃), 166.3 (s, C₃N₃), 165.7 (s, C₃N₃), 165.4 (s, C₃N₃), 165.2 (s, C₃N₃), 164.1 (s, C₃N₃), 156.2 (s, C(O)), 156.0 (s, C(O)), 79.3 (s, C(CH₃)₃), 72.2 (s, OCH₂CH₂OH), 70.1 (s, NHCH₂CH₂O), 61.7 (s, OCH₂CH₂OH), 47.9 (s, NCH₂), 47.4 (s, NCH₂), 43.5 and 43.1 (s, piperazine), 40.5 (s, NHCH₂CH₂O), 39.4 (s, CH₂NHBoc), 39.1 (s, CH₂NHBoc), 28.4 (s, C(CH₃)₃), 28.3 (s, C(CH₃)₃). MS (MALDI): calcd 2374.2210 (M⁺); found 2375.4347 (M+H⁺).

### Example 3

3: A clear solution of 6 (810 mg, 0.86 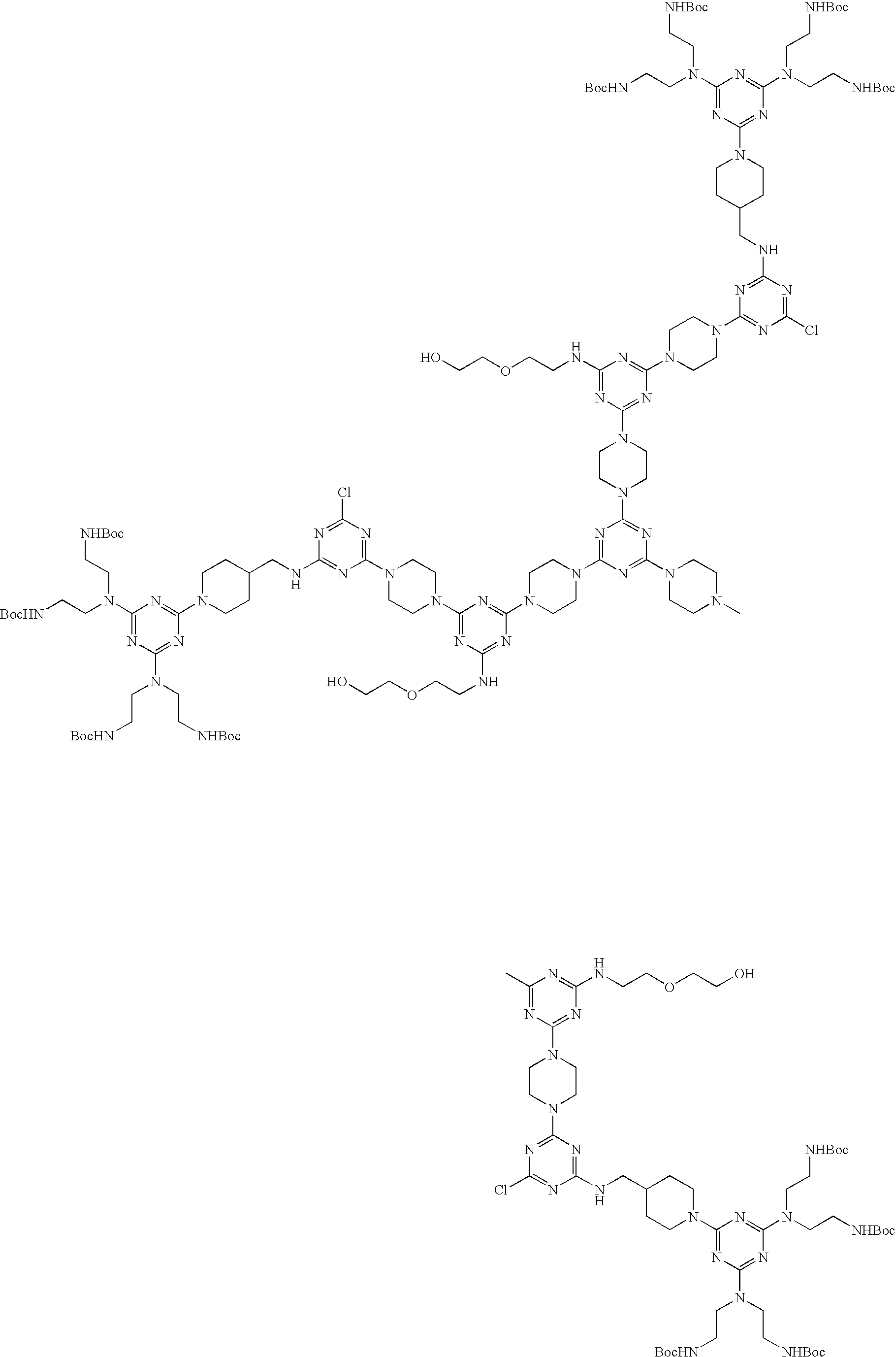
mmol) in 50 mL of THF was prepared, and a slurry of 10 (304 mg, 0.25 mmol) and DIPEA (0.4 mL, 2.35 mmol) was prepared in 50 mL of THF in a separate flask. Both solutions were cooled to 0° C., then combined. The mixture was warmed gradually to RT and stirred for 16 h, after which time the solvent was removed in vacuo. Purification was achieved using column chromatography on silica gel to afford the product as a white solid (20:1 CHCl₃:MeOH; R_{f}=0.33 using 10:1 CH₂Cl₂:MeOH as the developing solvent). Yield: 905 mg (96%). ¹H NMR (300 MHz, CDC1₃) □: 5.71 (br, 6H, NH), 5.59 (br, 6H, NH), 5.40 (br, 3H, NH), 5.23 (br, 3H, NH), 4.67 (d, ²J_{H—H}=12 Hz, 6H, amp-NCH₂), 3.79 (br, 54H, piperazine CH₂, OCH₂CH₂OH), 3.58 (br, 42H, CH₂, Boc-NCH₂), 3.30 (br, 30H, CH₂NHBoc, amp-CH₂), 2.76 (pseudo t, ²J_{H—H}=12 Hz, 6H, amp-NCH₂), 1.75 (br d, ²J_{H—H}=12 Hz, 6H, amp-NCH₂CH₂), 1.51 (m, 3H, amp-CH), 1.40 (s, 54H, C(CH₃)₃), 1.38 (s, 54H, C(CH₃)₃), 1.19 (m, 6H, amp-NCH₂CH₂). ¹³C{¹H} NMR (75.5 MHz, CDCl₃) □: 169.0 (s, C₃N₃), 166.2 (s, C₃N₃), 165.9 (s, C₃N₃), 165.8 (s, C₃N₃), 165.3 (s, C₃N₃), 165.1 (s, C₃N₃), 164.5 (s, C₃N₃), 164.2 (s, C₃N₃), 156.1 (s, C(O)), 79.2 (s, C(CH₃)₃), 79.0 (s, C(CH₃)₃), 72.2 (s, OCH₂CH₂OH), 70.0 (s, NHCH₂CH₂O), 61.7 (s, OCH₂CH₂OH), 47.3 (s, Boc-NCH₂), 46.6 (s, Boc-NCH₂), 46.4 (s, amp-NCH₂), 43.0 (s, CH₂, NHCH₂CH₂O and piperazine), 40.6 (s, amp-CH₂), 40.5 (s, CH₂NHBoc), 38.3 (s, CH₂NHBoc), 36.9 (s, amp-CH), 29.9, 29.7 (s, amp-NCH₂CH₂), 28.5 (s, C(CH₃)₃). MS (MALDI): calcd 3851.1728 (M⁺); found 3852.4626 (M+H⁺).

### Example 4

5: A solution of cyanuric chloride (1.17 g, 6.36 mmol) in THF (200 mL) was cooled to 0° C. A clear solution of HN(CH₂CH₂NHBoc)₂ (1.93 g, 6.36 mmol) in THF (20 mL) was added dropwise to the cyanuric chloride solution, followed by dropwise addition of a solution of DIPEA (1.3 mL, 7.63 mmol) in THF (20 mL). The solution was stirred at 0° C. for 1 h, then warmed gradually to RT and stirred for an additional 12 h. The solvent was removed in vacuo, and then the residue was taken up in CH₂Cl₂ (100 mL). The solution was washed with water (3×150 mL), and then dried with MgSO₄. Following filtration, the solvent was removed in vacuo. The product was obtained as a pure white solid by reprecipitation with hexanes from a clear solution of CH₂Cl₂. Yield: 2.79 g (97%). ¹H NMR (300 MHz, CDCl₃) □: 4.95 (br, 2H, NH), 3.72 (m, ³J_{H—H}=6 Hz, 4H, NCH₂), 3.39 (t, ³J_{H—H}=6 Hz, 2H, CH₂NHBoc), 3.37 (t, ³J_{H—H}=6 Hz, 2H, CH₂NHBoc), 1.37 (s, 18H, C(CH₃)₃). ¹³C{¹H} NMR (75.5 MHz, CDCl₃) □: 169.9 (s, C₃N₃), 165.6 (s, C₃N₃), 156.0 (s, C(O)), 79.5 (s, C(CH₃)₃), 48.6 (s, NCH₂), 38.5 (s, CH₂NHBoc), 28.2 (s, C(CH₃)₃). MS (ESI): calcd 450.1549 (M⁺); found 451.1528 (M+H⁺).

### Example 5

6: A solution of cyanuric chloride (655 mg, 3.52 mmol) in THF (75 mL) was cooled to 0° C. A clear solution of 16 (2.825 g, 3.55 mmol) in THF (100 mL) was added to the cyanuric chloride solution, followed by addition of a solution of DIPEA (0.73 mL, 4.52 mmol) in THF (20 mL). The solution was stirred at 0° C. for 30 min, then warmed gradually to RT and stirred for an additional 12 h. The solvent was removed in vacuo, and then the residue was taken up in CH₂Cl₂ (100 mL). The solution was washed with water (3×150 mL), and then dried with MgSO₄. Following filtration, the solvent was removed in vacuo. Purification was achieved using column chromatography on silica gel (3:1 CH₂Cl₂:EtOAc; R_{f}=0.08 using 10:1 CH₂Cl₂:EtOAc as the developing solvent) to afford the product as a white solid. Yield: 2.28 g (68%). ¹H NMR (300 MHz, CDCl₃) □: 5.62 (br, 2H, NH), 5.55 (br, 2H, NH), 4.69 (d, ²J_{H—H}=11 Hz, 2H, amp-NCH₂), 3.63 (br, 4H, Boc-NCH₂), 3.56 (br, 4H, Boc-NCH₂), 3.40 (br, 2H, amp-CH₂), 3.30 (br, 8H, CH₂NHBoc), 2.78 (pseudo t, ²J_{H—H}=11 Hz, 2H, amp-NCH₂), 1.70 (br d, ²J_{H—H}=13 Hz, 2H, amp-NCH₂CH₂), 1.4 (m, 1H, buried under ^{t}Bu proton peaks, amp-CH), 1.40 (s, 36H, C(CH₃)₃), 1.25 (m, 2H, amp-NCH₂CH₂). ¹³C{¹H} NMR (75.5 MHz, CDCl₃) □: 169.6 (s, C₃N₃), 166.0 (s, C₃N₃), 165.8 (s, C₃N₃), 164.2 (s, C₃N₃), 156.1 (s, C(O)), 79.1 (s, C(CH₃)₃), 78.9 (s, C(CH₃)₃), 47.2 (s, Boc-NCH₂), 46.7 (s, Boc-NCH₂), 46.5 (s, amp-NCH₂), 42.9 (s, amp-CH₂), 40.5 (s, CH₂NHBoc), 38.3 (s, CH₂NHBoc), 36.3 (s, amp-CH), 29.5 (s, amp-NCH₂CH₂), 28.4 (s, C(CH₃)₃). MS (MALDI): calcd 942.4722 (M⁺); found 943.5674 (M+H⁺).

### Example 6

8: Both 7 (423 mg, 1.27 mmol) and 4 (1.27 g, 3.8 mmol) were 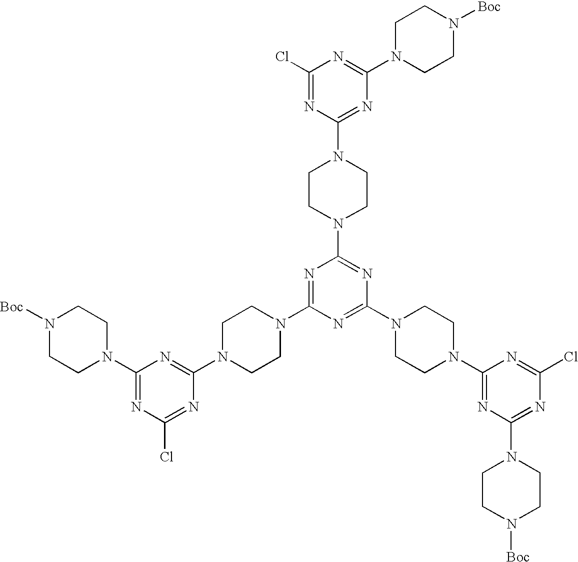
dissolved separately in THF (100 mL each) to give a slurry and a clear solution, respectively. DIPEA (2.15 mL, 12.7 mmol) was added to the solution of 7, and then both solutions were cooled to 0° C. The two solutions were combined rapidly, and the slurry was stirred at 0° C. for 1 h. The solution was then warmed gradually to RT and stirred for an additional 15 h. The solvent was removed in vacuo. The residue was dissolved in CH₂Cl₂ (100 mL), and this solution was washed with water (3×100 mL). The organic phase was dried with MgSO₄, and following filtration, the solvent was removed in vacuo. Purification was achieved using column chromatography on silica gel (4:1 CH₂Cl₂:EtOAc; R_{f}=0.13 using 10:1 CH₂Cl₂:EtOAc as the developing solvent) to afford the product as a white solid. Yield: 1.29 g (83%). ¹H NMR (300 MHz, CDCl₃) □: 3.77 (br, 36H, CH₂, piperazine), 3.41 (br, 12H, CH₂NBoc), 1.42 (s, 27H, C(CH₃)₃). ¹³C{¹H} NMR (75.5 MHz, CDCl₃) □: 169.5 (s, C₃N₃), 165.1 (s, C₃N₃), 164.3 (s, C₃N₃), 154.5 (s, C(O)), 80.1 (s, C(CH₃)₃), 43.2 (s, CH₂), 43.1 (s, CH₂), 42.9 (s, CH₂NBoc), 42.6 (s, CH₂NBoc), 28.2 (s, C(CH₃)₃). MS (MALDI): calcd 1224.5367 (M⁺); found 1225.7524 (M+H⁺).

### Example 7

9: A clear solution of 8 (968 mg, 0.76 mmol), 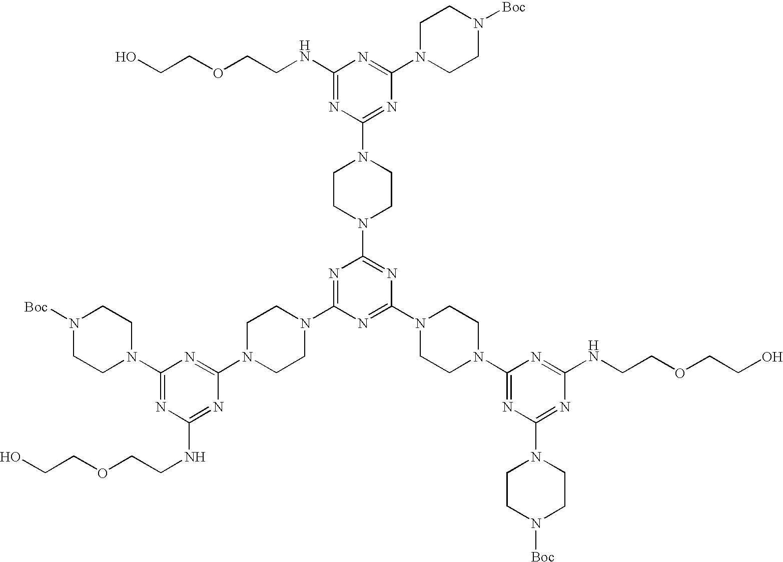
DIPEA (0.4 mL, 2.4 mmol) and H₂NCH₂CH₂OCH₂CH₂OH (0.8 mL, 8.0 mmol) in THF (25 mL) was heated at 65° C. for 24 h, during which time a precipitate formed. The solvent was removed in vacuo. The residue was dissolved in CH₂Cl₂ (100 mL), and this solution was washed with with water (3×150 mL). The organic phase was dried with MgSO₄, and following filtration, the solvent was removed in vacuo to afford the product as a white solid (1.06 g, 97%). ¹H NMR (300 MHz, CDCl₃) □: 5.16 (br t, 3H, NH), 3.78 (br, 36H, CH₂, piperazine), 3.72 (br, 6H, CH₂), 3.59 (br, 18H, CH₂), 3.42 (br, 12H, CH₂NBoc), 2.87 (br t, 3H, OH), 1.46 (s, 27H, C(CH₃)₃). ¹³C{¹H} NMR (75.5 MHz, CDCl₃) □: 166.2 (s, C₃N₃), 165.3 (s, C₃N₃), 165.1 (s, C₃N₃), 154.8 (s, C(O)), 79.9 (s, C(CH₃)₃), 72.3 (s, OCH₂CH₂OH), 70.1 (s, NHCH₂CH₂O), 61.6 (s, OCH₂CH₂OH), 43.0 and 42.9 (s, CH₂CH₂NBoc, piperazine), 40.4 (s, NHCH₂CH₂O), 28.4 (s, C(CH₃)₃). MS (MALDI): calcd 1431.8436 (M⁺); found 1432.9504 (M+H⁺).

### Example 8

10: Concentrated aqueous HCl (20 mL) was 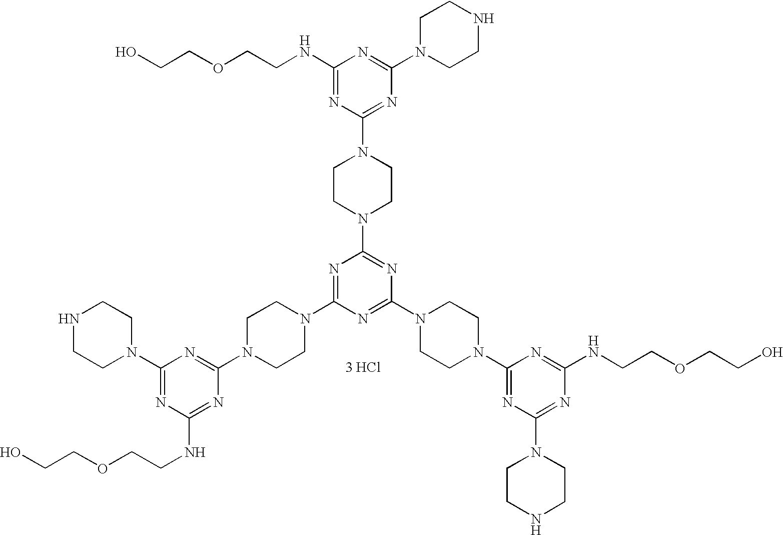
added to a slurry of 9 (1.01 g, 0.71 mmol) in MeOH (40 mL). The solution grew clear, and was stirred at RT for 12 h. Alternatively, the solution could be heated at 50° C. for 3 h to decrease the reaction time. Following either of these protocols, the solution was concentrated in vacuo until only ca. 5 mL of water remained. The residue was diluted with 50 mL of water, and this solution was washed with CH₂Cl₂ (3×100 mL). The solvent was removed from the aqueous layer in vacuo to afford the product as a white solid. Yield: 838 mg, 96% (calculated assuming 3 HCl salt). Note that to determine complete deprotection of the Boc groups on the precursor molecule occurred, MS analysis must be performed to ensure none of the possible by-products exist since they are not detectable in low amounts using ¹H or ¹³C{¹H} NMR spectroscopy or TLC analysis. ¹H NMR (300 MHz, D₂O) □: 4.14 (br, 12H), 3.95 (br, 24H), 3.69 (br, 18H), 3.64 (br, 6H), 3.34 (br, 12H). ¹³C{¹H} NMR (75.5 MHz, D₂O) □: 161.9 (s, C₃N₃), 158.1 (s, C₃N₃), 156.0 (s, C₃N₃), 154.3 (s, C₃N₃), 71.7 (s, OCH₂CH₂OH), 68.6 (s, NHCH₂CH₂O), 60.5 (s, OCH₂CH₂OH), 43.5 (br, CH₂, piperazine), 43.1 (br, CH₂, piperazine), 40.7 (br, CH₂, piperazine), 40.3 (s, NHCH₂CH₂O). MS (MALDI): calcd 1131.6863 (M⁺); found 1132.8062 (M+H⁺).

### Example 9

14: A solution of cyanuric chloride (649 mg, 3.52 mmol) in THF (75 mL) was cooled to 0° C. A clear solution of 13 (2.34 g, 7.70 mmol) in THF (100 mL) was added to the cyanuric chloride solution, followed by addition of a solution of DIPEA (1.8 mL, 10.6 mmol) in THF (20 mL). The solution was stirred at 0° C. for 30 min, then warmed gradually to RT and stirred for an additional 12 h. The solvent was removed in vacuo, and then the residue was taken up in CH₂Cl₂ (100 mL). The solution was washed with water (3×150 mL), and then dried with MgSO₄. Following filtration, the solvent was removed in vacuo. The product was obtained as a pure white solid by reprecipitation with hexanes from a clear solution of CH₂Cl₂. Yield: 2.52 g (99%). ¹H NMR (300 MHz, CDCl₃) □: 5.53 (br, 2H, NH), 5.15 (br, 2H, NH), 3.64 (t, ³J_{H—H}=6 Hz, 4H, NCH₂), 3.58 (t, ³J_{H—H}=6 Hz, 4H, NCH₂), 3.28 (m, 8H, CH₂NHBoc), 1.40 (s, 18H, C(CH₃)₃), 1.38 (s, 18H, C(CH₃)₃). ¹³C{¹H} NMR (75.5 MHz, CDCl₃) □: 168.7 (s, C₃N₃), 165.1 (s, C₃N₃), 156.1 (s, C(O)), 79.0 (s, C(CH₃)₃), 47.8 (s, NCH₂), 47.0 (s, NCH₂), 39.3 (s, CH₂NHBoc), 37.4 (s, CH₂NHBoc), 28.3 (s, C(CH₃)₃), 28.2 (s, C(CH₃)₃). MS (ESI): calcd 717.3940 (M⁺); found 718.5813 (M+H⁺).

### Example 10

15: Neat 4-aminomethylpiperidine (1 mL, 7 mmol) was added to 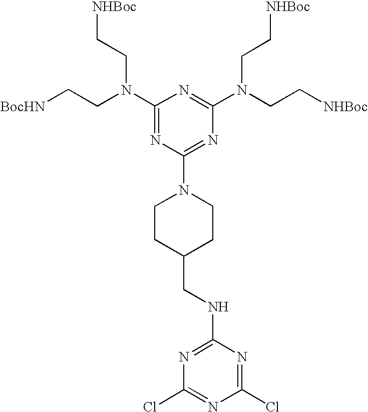
a clear solution of 14 (849 mg, 1.18 mmol) in THF (80 mL). The solution was stirred at RT for 12 h. The solvent was removed in vacuo, and then the residue was taken up in CH₂Cl₂ (100 mL). The solution was washed with water (3×150 mL), and then dried with MgSO₄. Following filtration, the solvent was removed in vacuo to afford the product as a pure white foam. Yield: 940 mg (99%). ¹H NMR (300 MHz, CDCl₃) □: 5.77 (br, 2H, NH), 5.53 (br, 2H, NH), 4.68 (d, ²J_{H—H}=13 Hz, 2H, amp-NCH₂), 3.64 (br, 4H, Boc-NCH₂), 3.55 (br, 4H, Boc-NCH₂), 3.30 (br, 8H, CH₂NHBoc), 2.76 (pseudo t, ²J_{H—H}=13 Hz, 2H, amp-NCH₂), 2.57 (d, ³J_{H—H}=6 Hz, 2H, amp-CH₂), 1.76 (br d, ²J_{H—H}=11 Hz, 2H, amp-NCH₂CH₂), 1.4 (m, 1H, buried under ^{t}Bu proton peaks, amp-CH), 1.40 (s, 18H, C(CH₃)₃), 1.38 (s, 18H, C(CH₃)₃), 1.11 (q of d, ²J_{H—H}=11 Hz, ³J_{H—H}=4 Hz, 2H, amp-NCH₂CH₂). ¹³C{¹H} NMR (75.5 MHz, CDCl₃) □: 165.8 (s, C₃N₃), 164.0 (s, C₃N₃), 156.1 (s, C(O)), 156.0 (s, C(O)), 78.8 (s, C(CH₃)₃), 78.6 (s, C(CH₃)₃), 47.7 (s, Boc-NCH₂), 47.0 (s, Boc-NCH₂), 46.2 (s, amp-NCH₂), 43.0 (s, amp-CH₂), 40.6 (s, CH₂NHBoc), 39.7 (s, amp-CH), 38.1 (s, CH₂NHBoc), 29.6 (s, amp-NCH₂CH₂), 28.2 (s, C(CH₃)₃). MS (MALDI): calcd 795.5331 (M⁺); found 796.5796 (M+H⁺).

### Example 11

The following synthetic scheme outlines an alternative means of synthesizing macromolecules of the present invention.

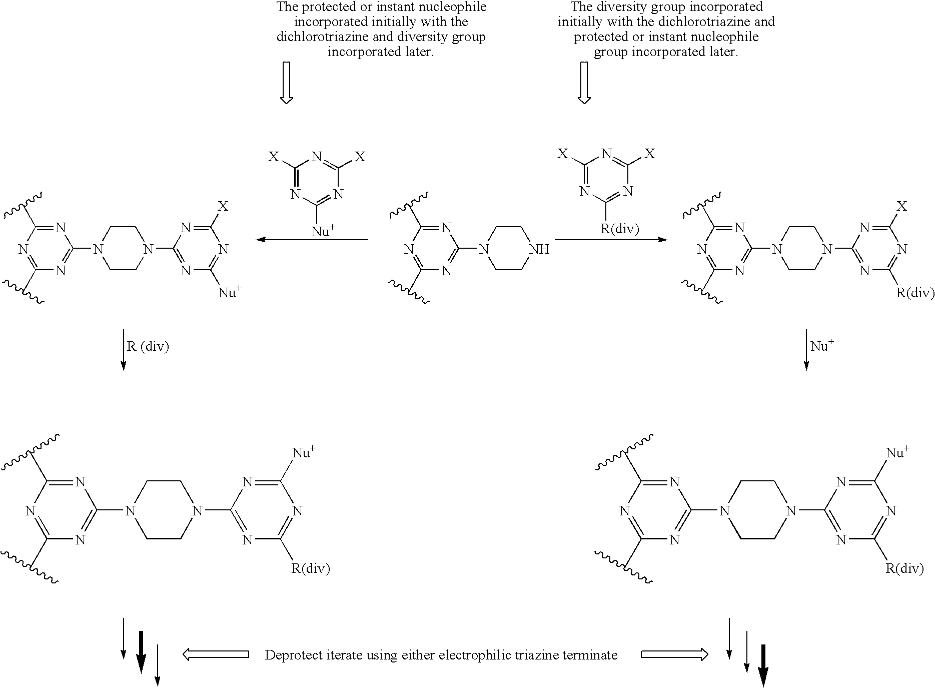

All of the compositions and/or methods disclosed and claimed in this specification can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 6,692,724
“Protective Groups in Organic Synthesis”, 3^{rd} ed., by T. W. Greene and P. G. M. Wuts, John Wiley & Sons, New York, N.Y. (1999) Chapter 6.

What is claimed is:

1. A method of synthesizing a macromolecule, comprising:
(a) obtaining a nucleophile-bearing core including at least one nucleophilic group, and
(b) reacting said core with a first monomeric electrophilic triazine including at least one substitutable group and at least one of either a protected-nucleophilic group or an unreactive group, wherein said macromolecule has one less substitutable group when compared to said first monomeric electrophilic triazine and at least one of either a protected-nucleophilic group or unreactive group.

2. The method of claim 1, wherein the first monomeric electrophilic triazine includes at least 2 substitutable groups, such that the macromolecule includes at least one substitutable group.

3. The method of claim 2, further comprising reacting the macromolecule with at least one substitutable group with a first diversity group, such that at least one substitutable group is substituted with the first diversity group.

4. The method of claim 3, further comprising removing at least one protecting group of the macromolecule.

5. The method of claim 2, further comprising reacting the macromolecule with a second electrophilic monomeric triazine including at least one substitutable group and at least one of either a protected-nucleophilic group or an unreactive group, wherein said macromolecule has one less substitutable group when compared to said second monomeric electrophilic triazine and at least one of either a protected-nucleophilic group or unreactive group.

6. The method of claim 5, further comprising removing one or more protecting groups.

7. The method of claim 4, further comprising subjecting the macromolecule to iterative steps of claim 1, wherein the macromolecule becomes the nucleophile-bearing core of step (a).

8. The method of claim 6, further comprising subjecting the macromolecule to iterative steps of claim 1, wherein the macromolecule becomes the nucleophile-bearing core of step (a).

9. The method of claim 1, wherein the first monomeric electrophilic triazine includes at least one unreactive group.

10. The method of claim 1, wherein the first monomeric, electrophilic triazine further comprises at least one diversity group and two substitutable groups.

11. The method of claim 10, further comprising reacting the macromolecule with a nucleophile-bearing group, wherein said macromolecule has one less substitutable group when compared to said first monomeric electrophilic triazaine.

12. The method of claim 11, further comprising removing one or more protecting groups from the macromolecule.

13. The method of claim 1, wherein the first monomeric electrophilic triazine includes at least one nucleophile-bearing group and at least two substitutable groups.

14. The method of claim 13, further comprising reacting the macromolecule with a diversity group, wherein said macromolecule has one less substitutable group when compared to said first monomeric electrophilic triazaine.

15. The method of claim 14, further comprising removing one or more protecting groups from the macromolecule.

16. The method of claim 12, further comprising subjecting the macromolecule to iterative steps of claim 11, wherein the macromolecule becomes the nucleophile-bearing group.

17. The method of claim 15, further comprising subjecting the macromolecule to iterative steps of claim 10.4, wherein the macromolecule becomes the nucleophile-bearing core.

18. The method of claim 12, further comprising subjecting the macromolecule to iterative steps of claim 14, wherein the macromolecule becomes the nucleophile-bearing group.

19. The method of claim 15, further comprising subjecting the macromolecule to iterative steps of claim 11, wherein the macromolecule becomes the nucleophile-bearing core.

20. The method of claim 1, wherein the first monomeric electrophilic triazine includes at least 2 or at least 4 nucleophilic groups.

21. The method of claim 1, wherein the nucleophile-bearing core including at least one nucleophilic group comprises: 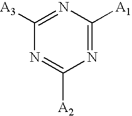
wherein:
A₁ is a first nucleophile-bearing group and,
A₂ and A₃ are selected from a group consisting of a second nucleophile-bearing group and a third nucleophilic group, and an unreactive group.

22. The method of claim 21, wherein any one or more of A₁-A₃ is an amine-bearing group.

23. The method of claim 21, wherein the amine-bearing group is selected from a group consisting of an —NH₂-bearing group and an —R—NH₂-bearing group, wherein R is selected from the group consisting of a hydrocarbon-containing group, a secondary or tertiary amine-containing group, an oxo-containing group, a thiol-containing group, an amido-containing group, and any combination of one or more of these groups.

24. The method of claim 21, wherein the amine-bearing group is a secondary amine-bearing group.

25. The method of claim 24, wherein the secondary amine-bearing group is a cycloalkylamino-bearing group.

26. The method of claim 25, wherein the cycloalkylamino-bearing group is selected from the group consisting of a piperazino-bearing 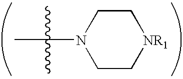
group and a (R₁-aminoalkyl)cycloamino-bearing group, wherein R₁ equals H, R-amino, acyl, or triazinyl and wherein R₁ is selected from the group consisting of a hydrocarbon-containing group, a secondary or tertiary amine-containing group, an oxo-containing group, a thiol-containing group, an amido-containing group, and any combination of one or more of these groups.

27. The method of claim 21 wherein any one or more of A₁-A₃ is selected from a group consisting of a sulfur group in a reactive state or an oxygen group in a reactive state.

28. The method of claim 21, wherein the nucleophile-bearing group of A₁ and A₂ are the same.

29. The method of claim 21, wherein each of the nucleophile-bearing groups of A₁ and A₂ is different.

30. The method of claim 21, wherein A₁, A₂, and A₃ are all nucleophile-bearing groups that are the same.

31. The method of claim 21, wherein A₁, A₂, and A₃ are all nucleophile-bearing groups that are different.

32. The method of claim 21, wherein the unreactive group is selected from the group consisting of a hydrocarbon-containing group, a secondary or tertiary amine-containing group, an oxo-containing group, a thiol-containing group, an amido-containing group, and any combination of one or more of these groups.

33. The method of claim 1, wherein one or more of the monomeric electrophilic triazines comprises: 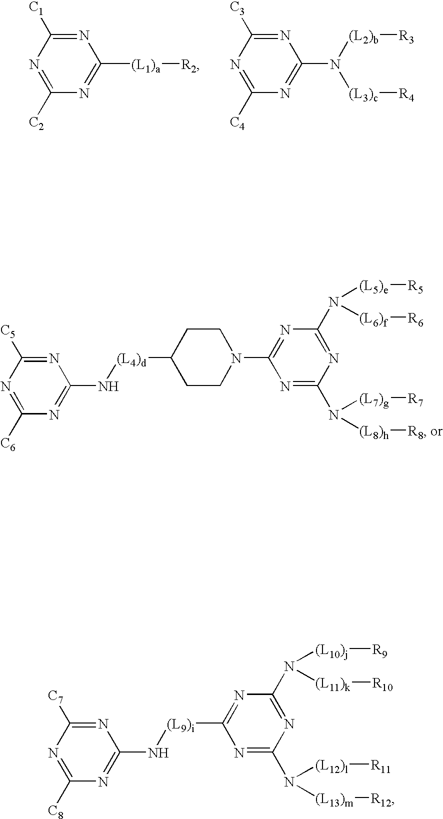
wherein:
C₁-C₈ is each independently a substitutable group,
L₁-L₁₃ is each independently a linker group,
R₂-R₁₂ is each independently a protected nucleophilic group, and
a-m is each independently 0-200.

34. The method of claim 33, wherein the any one or more of C₁-C₈ is a halogen.

35. The method of claim 34, wherein the halogen is chlorine.

36. The method of claim 33, wherein any one or more of L₁-L₁₃ is selected from the group consisting of a hydrocarbon-containing group, a secondary or tertiary amine-containing group, an oxo-containing group, a thiol-containing group, an amido-containing group, and any combination of one or more of these groups.

37. The method of claim 36, wherein the secondary or tertiary amine-containing group is piperazino- 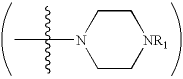
wherein R₁ equals H, R-amino, acyl, or triazinyl, and wherein R₁ is selected from the group consisting of a hydrocarbon-containing group, a secondary or tertiary amine-containing group, an oxo-containing group, a thiol-containing group, an amido-containing group, and any combination of one or more of these groups.

38. The method of claim 37, wherein one or more oxo-containing groups is an ether.

39. The method of claim 38, wherein the ether is polyethylene glycol.

40. The method of claim 33, wherein any one or more of L₁-L₁₃ comprises one or more amido-containing groups.

41. The method of claim 40, wherein the one or more amido-containing groups is a protected carbohydrate or a protected peptide.

42. The method of claim 33, wherein any one or more of L₁-L₁₃ comprises a hydrocarbon group.

43. The method of claim 42, wherein the one or more alkyl groups comprises (—CH₂—)ₐ₋ₘ.

44. The method of claim 43, wherein any one or more of (—CH₂—)ₐ₋ₘ groups is an ethyl or propyl group.

45. The method of claim 24, wherein any one or more of R₂—R₁₂ is selected from the group consisting of a protected amino group, a protected hydroxyl group, a protected carbonyl group, a protected carboxyl group, a protected thiol group, and a protected phosphate group.

46. The method of claim 45, wherein any one or more of R₂-R₁₂ comprises a protected amino group.

47. The method of claim 46, wherein the protected amino group is selected from a group consisting of a Boc-protected amino group or an acetyl protected amino group.

48. The method of claim 24, wherein any one or more -(L₁₋₁₃)ₐ₋ₘ-R₂₋₁₂) comprises one or more ethylamino groups wherein one or more of the amino groups is protected by a protecting group.

49. The method of claim 48, wherein the one or more protecting groups is selected from a group consisting of a Boc group or an acetyl group.

50. The method of claim 46, wherein protected amino group provides a protecting group that can be readily converted to a nucleophilic amine in one or more steps.

51. The method of claim 50, wherein the protecting group that can be readily converted to a nucleophilic amine in one or more steps is selected from a group consisting of a carbamate, an amide, and an imide.

52. The method of claim 51, wherein the carbamate is BOC.

53. The method of claim 51, wherein the amide is acetyl.

54. The method of claim 51, wherein the imide is phthalamidoyl.

55. The method of claim 33, wherein any one or more of R₂-R₁₂ comprises a protected hydroxyl group.

56. The method of claim 33, wherein a-m is each independently 0-10.

57. The method of claim 56, wherein a-m is each independently 0-2.

58. The method of claim 3, wherein the diversity group is selected from the group consisting of H, a hydrogen-containing group, a carbon-containing group, a secondary or tertiary amine-containing group, an oxo-containing group, a thiol-containing group, an amido-containing group, a phosphino-containing group, a metal-containing group, a nucleophile-bearing group, a protected nucleophile-bearing group, an electrophile-bearing group, a compatibilizing group, a polymer, a resin, a bead, a targeting group, a drug, a solubility enhancer, and any combination of one or more of these groups.

59. The method of claim 58, wherein the diversity group is a carbon-containing group attached to the macromolecule.

60. The method of claim 59, wherein the carbon-containing group is an electrophile-bearing group.

61. The method of claim 59, wherein the carbon-containing group is a nucleophile-bearing group.

62. The method of claim 59, wherein the diversity group is attached to the macromolecule through an attachment consisting of the group selected from carbon—carbon single bond, an alkene, an amide, a sulfonamide, an ester, an ether, a thioether, a carbonyl, and a thiocarbonyl.

63. The method of claim 58, wherein the diversity group is —(CH₂)₂—O—(CH₂)ₙ—OH or —(CH₂)₂—O—(CH₂)ₙ—OCH₃.

64. The method of claim 58, wherein the diversity group is selected from a group consisting of a protein, a peptide, a carbohydrate, an enzyme, an antibody, an antibacterial agent, an antibiotic, an antiviral agent, an antifungal agent, an anticancer agent, a tumor marker, a cell targeting ligand, a DNA intercalator, an organ-specific ligand, and a compatibilizing group.

65. The method of claim 64, wherein the compatibilizing group is selected from the group consisting of an anionic group, a cationic group, or a polyalkylene glycol.

66. The method of claim 65, wherein the polyalkylene glycol is polyethylene glycol.

67. The method of claim 58, wherein the diversity group is a targeting group.

68. The method of claim 67, wherein the targeting group is a therapeutic agent.

69. The method of claim 3, wherein the diversity group is attached to the macromolecule via a linker.

70. The method of claim 69, wherein the linker is selected from the group consisting of a hydrocarbon-containing group, a secondary or tertiary amine-containing group, an oxo-containing group, a thiol-containing group, an amido-containing group, a phosphino-containing group, and any combination of one or more of these groups.

71. The method of claim 69, wherein the linker is selected from the group consisting of

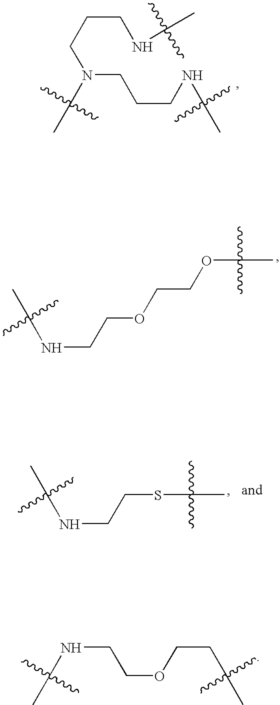

72. A macromolecule compound synthesized by any one of the methods of claims 1 to 71.

73. The macromolecule compound of claim 72 selected from the group consisting of:

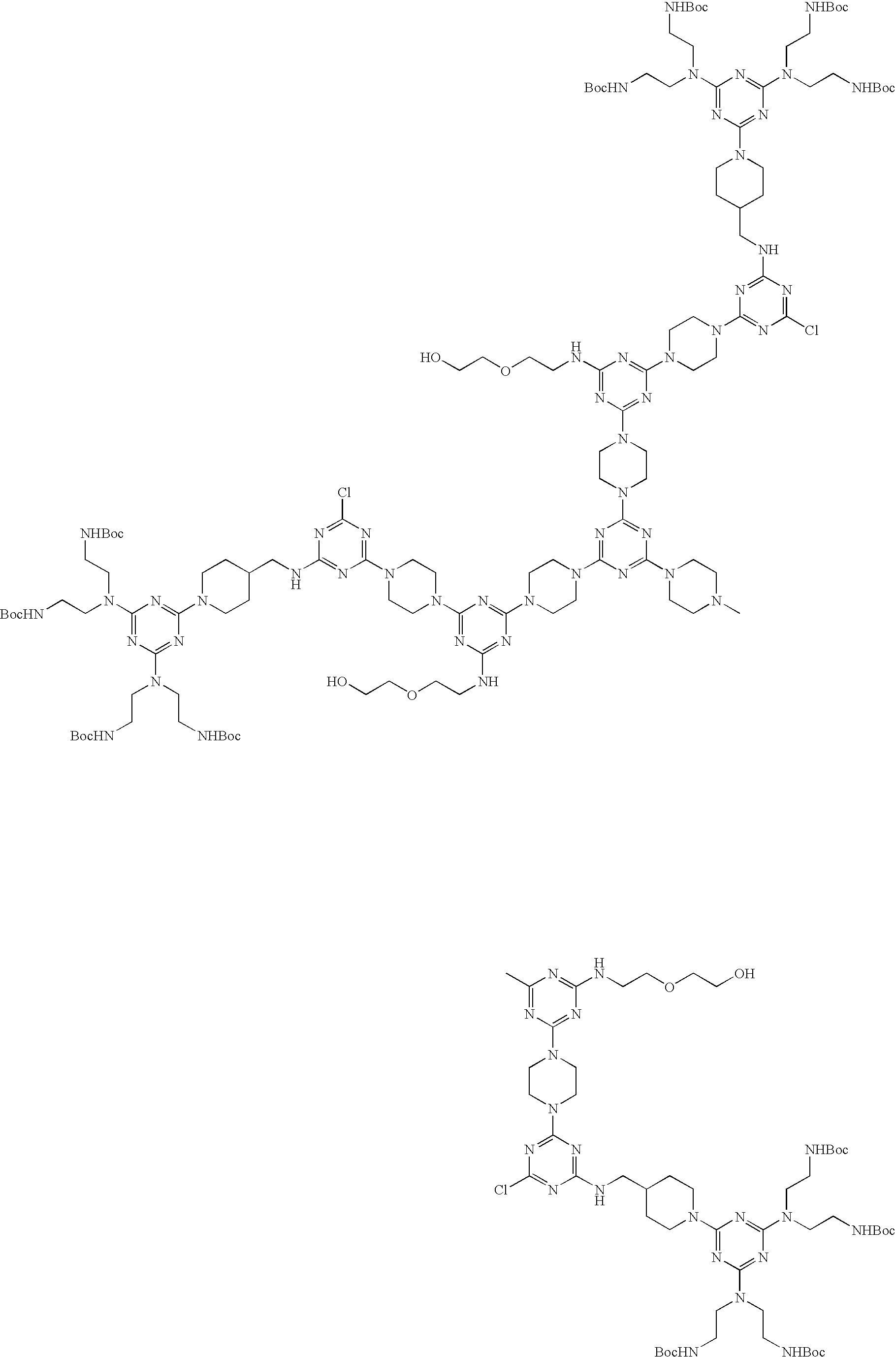

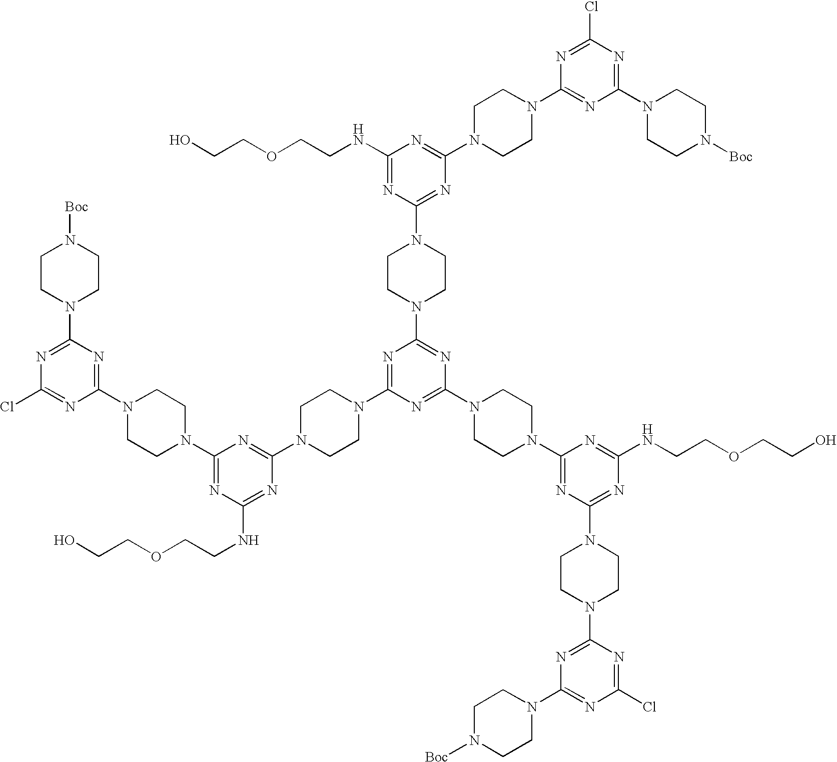

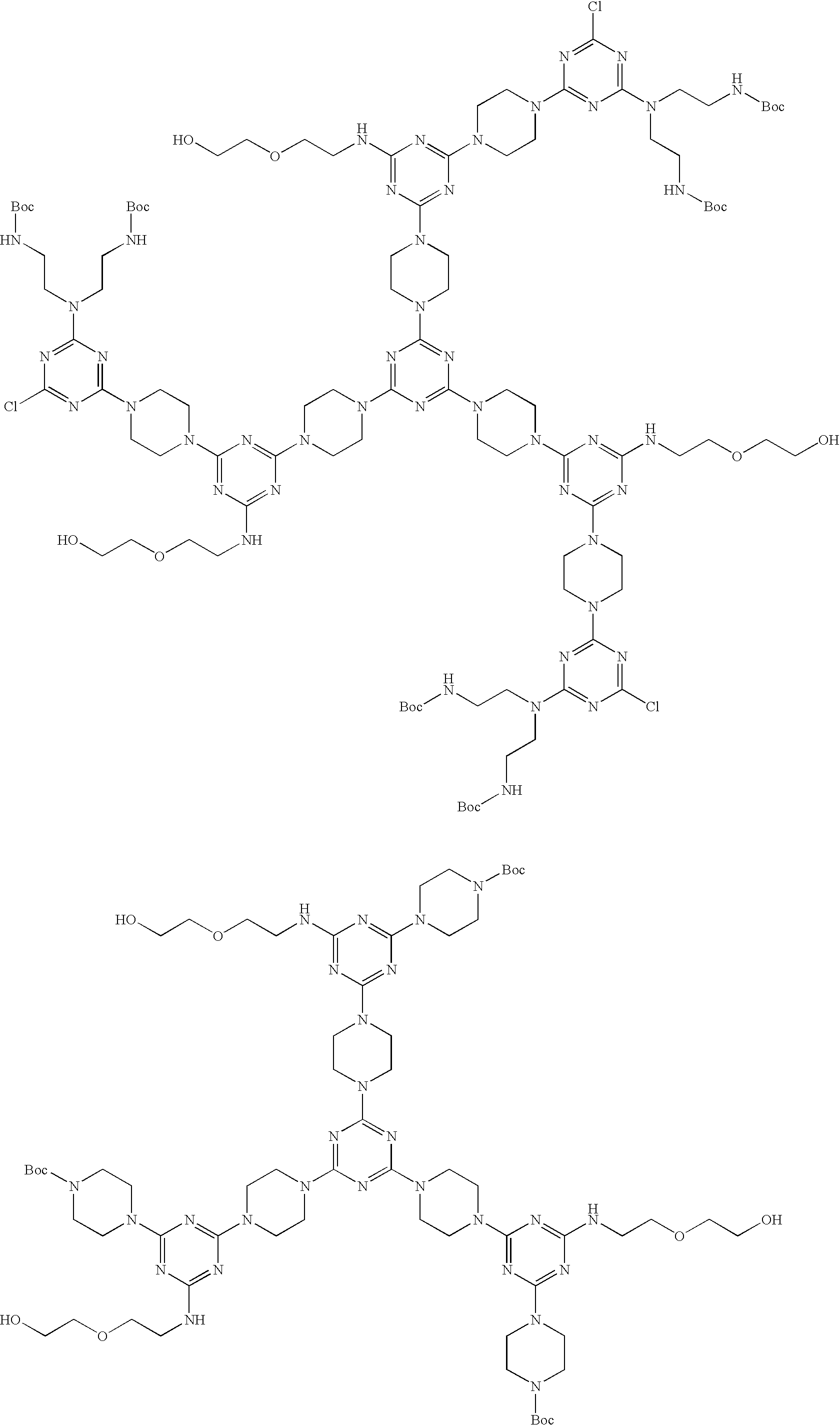

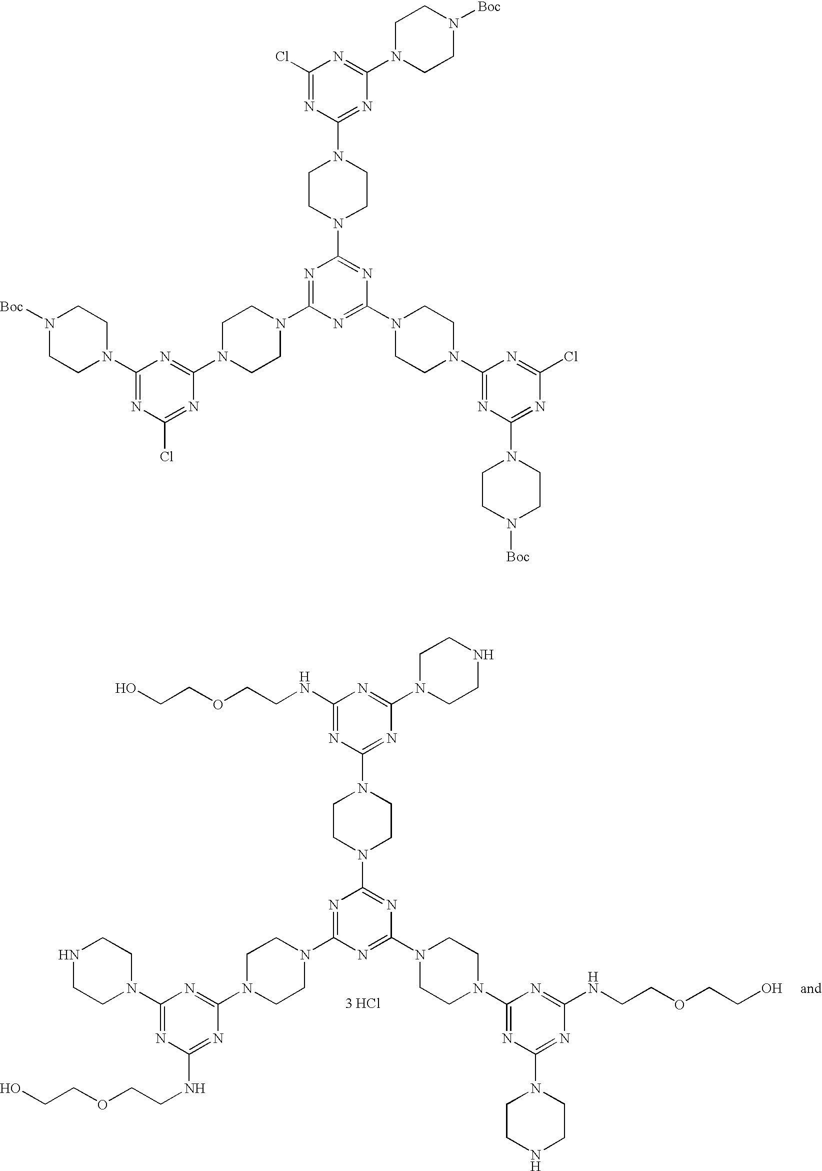

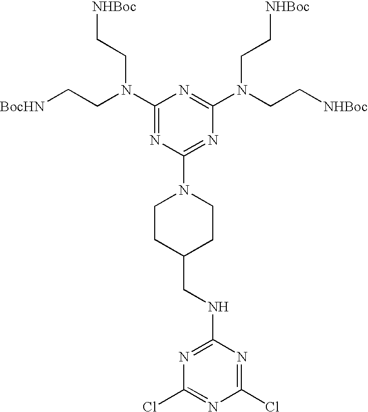

74. A monomeric electrophilic triazine compound of the formula: 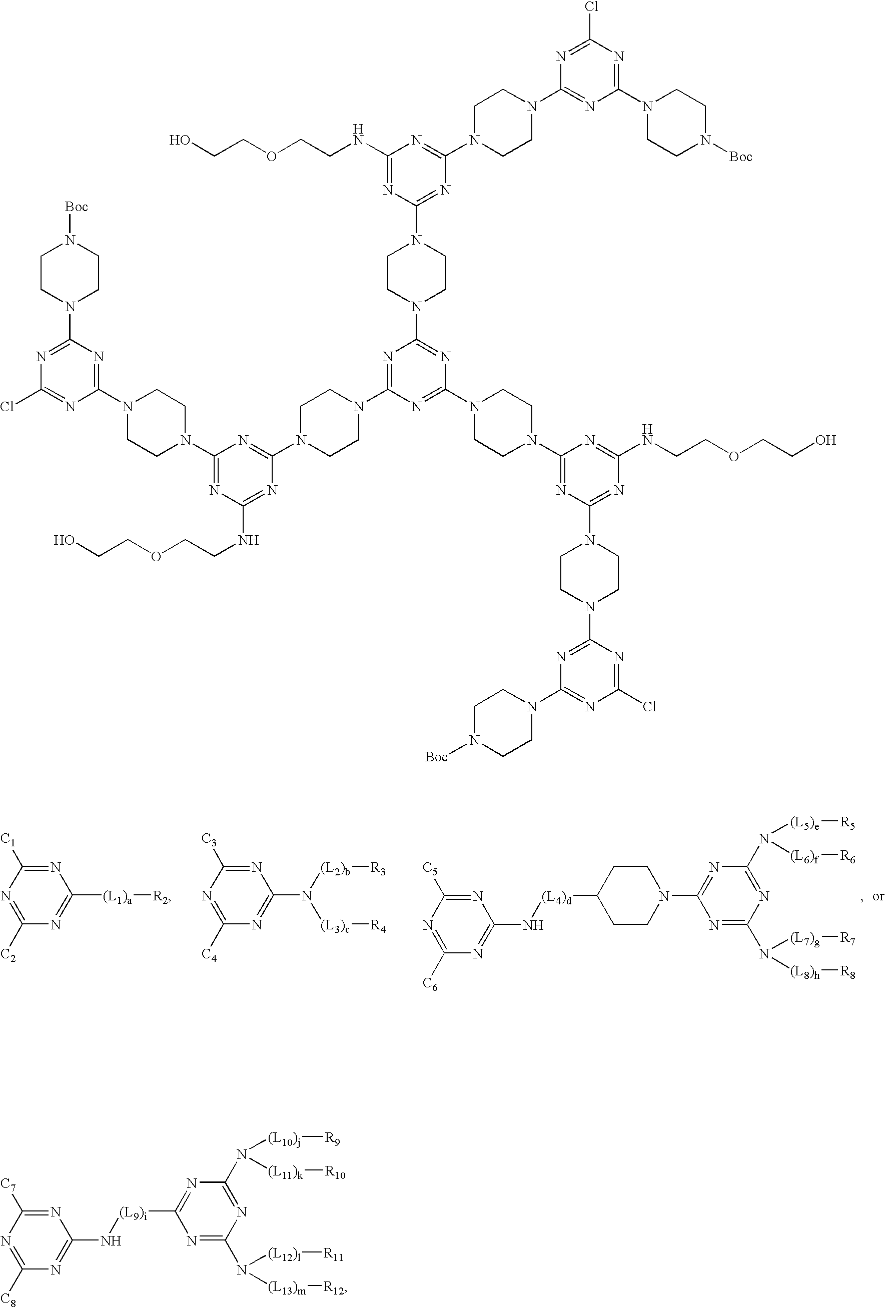
wherein:
C₁-C₈ is each independently a substitutable group,
L₁-L₁₃ is each independently a linker group,
R₂-R₁₂ is each independently a protected nucleophilic group, and
a-m is each independently 0-200.

75. A compound of the formula: H-J-K, wherein:
H comprises a nucleophile-bearing core having one or more nucleophilic groups of the formula: 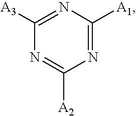
wherein:
A₁ is a first nucleophile-bearing group, and
A₂, and A₃ are selected from a group consisting of a second nucleophile-bearing group, which may or may not be the same as the first nucleophilic group, a third nucleophilic group, which may or may not be the same as the first and second nucleophilic groups, and an unreactive group;
provided that H is bound to J through a nucleophilic reaction wherein one of H's nucleophilic groups has reacted with one of J's electrophilic groups;
J comprises a monomeric electrophilic triazine of the formula: 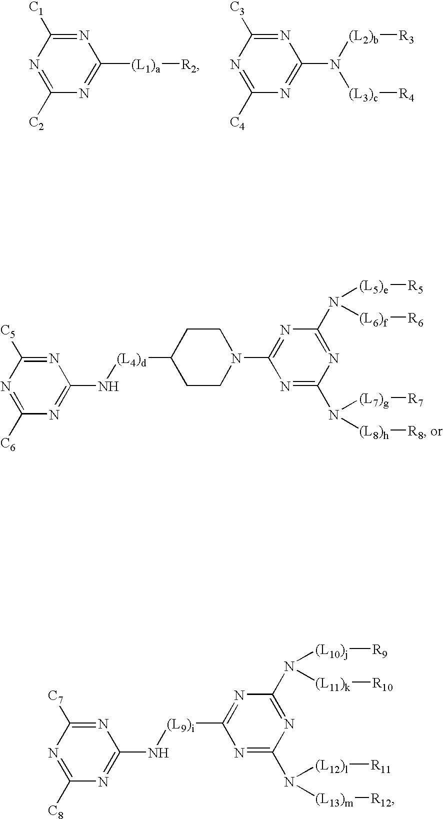
wherein:
C₁-C₈ is each independently a substitutable group,
L₁-L₁₃ is each independently a linker group,
R₂-R₁₂ is each independently a protected nucleophilic group, and a-m is each independently 0-200.
provided that J is bound to H in the manner described above and one bond of J is bound to K via a covalent bond; and
K comprises a diversity group.

76. A method of treating, diagnosing, or imaging a subject, comprising administering to the subject a pharmaceutically effective amount of a macromolecule prepared by the method of anyone of claims 1 through 71, or a compound of claim 74 or 75.

77. The method of claim 76, wherein the subject is selected from the group consisting of a receptor, cell, tissue, organ, or mammal.

78. The method of claim 76, wherein the macromolecule is the compound of claim 74.

79. The method of claim 76, wherein the macromolecule is the compound of claim 75.
